(19) United States
(12) Patent Application Publication
(10) Pub. No.: US 2023/0073781 A1
(43) Pub. Date: Mar. 9, 2023

(54) Systemic administration of peptides for the treatment of spinal cord injury and/or for remyelination

(71) Applicant: AXOLTIS PHARMA, CLERMONT-FERRAND (FR)

(72) Inventors: Yann GODFRIN, CALUIRE (FR); Manuel BLANC, LYON (FR)

(21) Appl. No.: 17/767,099

(22) PCT Filed: Oct. 7, 2020
(86) PCT No.: PCT/EP2020/078127

§ 371 Date: Apr. 7, 2022

(30) Foreign Application Priority Data
Oct. 7, 2019 (EP) 19306299.9

(51) Int. Cl. C07K 14/47; A61P 25/00; A61P 25/28

(52) U.S. Cl. CPC C07K 14/47; A61P 25/00; A61P 25/28; A61K 38/00

(57) ABSTRACT

The invention concerns treating non-brain nervous system injury, such as spinal cord injury and/or optic nerve injury, with a SCO-Spondin derived peptide administered through a systemic route to the patient. Said peptide has amino acid sequence X1-W-S-A1-W-S-A2-C-S-A3-A4-C-G-X2, in which A1, A2, A3 and A4 consists of amino acid sequences consisting of 1 to 5 amino acids, X1 and X2 consists of amino acid sequences consisting of 1 to 6 amino acids; or X1 and X2 are absent; it being possible for the N-terminal amino acid to be acetylated, for the C-terminal amino acid to be amidated, or the N-terminal amino acid to be acetylated and the C-terminal amino acid to be amidated. Also described the use of such peptides for remyelination.

The present invention relates to the systemic administration of SCO-Spondin derived peptides for the treatment of non-brain nervous system injury, such as spinal cord injury and/or optic nerve injury. It also relates to the systemic administration of SCO-Spondin derived peptides for the remyelination in myelopathy, including spinal cord injury and the other forms of myelopathy related to the spinal cord or the central nervous system.

### BACKGROUND OF INVENTION

Spinal cord injury (SCI) results in high morbidity with incomplete/complete sensory-motor paralysis. The primary injury leads to necrosis and bleeding, followed by secondary injuries including gliosis.

Traumatic optic nerve injury is a leading cause of irreversible blindness across the world and causes progressive visual impairment. To date, neither pharmacological nor surgical interventions are sufficient to halt or reverse the progress of visual loss. Axon regeneration is critical for functional recovery of vision following optic nerve injury. Optic nerve injury can be caused by primary and secondary mechanisms. Primary injury is by permanent axonal injury at the moment of impact from mechanical shearing, contusion, and ischemic necrosis of nerve axons. Secondary mechanisms are due to apoptosis, edema, and cell death, incorporating a variety of mechanisms leading to further axonal damage after the initial impact (Schmidek and Sweet, Operative Neurosurgical Techniques (Sixth Edition), 2012, Pages 2329-2338). The nervous system is divided into two parts: the peripheral nervous system and the central nervous system. If damaged peripheral nerves can regenerate after injury, various inhibitory factors however impede optic nerve and spinal cord regeneration after injury. There are thus important unmet medical needs for the treatment of spinal cord and/or optic nerve injury.

Spinal Cord and Optic Nerve are surrounded by Cerebrospinal fluid (CSF) and isolated from the blood by the Blood Brain Barrier (BBB), the blood Spinal Cord barrier and the blood Optic nerve barrier notably (hereinafter called “blood barriers”).

Due to their safety, target specificity and potency, peptides are increasingly becoming a therapeutic modality of choice for the development of treatments in many disease settings. One of the major limitations of therapeutic peptides is their short half-life which results in a low bio-availability. In the case of the development of therapeutic peptides for CNS conditions another important problem is their low penetration across biological barriers such as the Blood Brain Barrier (BBB), the blood Spinal Cord barrier, and the blood Optic nerve barrier notably.

SCO-Spondin derived peptides have been described for their neuroregenerative properties notably their ability to improve cell survival and neurite outgrowth in vitro. NX210 SCO-Spondin derived peptide has been shown to improve axonal regrowth in a rat model of SCI when administered directly at the lesion site in a collagen tube (Sakka et al. 2014, Plos One 9(3): e93179). The same authors have shown that NX210 significantly improves functional recovery in a rat contusion model of SCI when administered into the lesion.

### SUMMARY OF INVENTION

The inventors made the unexpected finding that SCO-Spondin derived peptides could be administered via the systemic route and yet retain efficacy for the treatment of non-brain nervous system injury, such as spinal cord injury and optic nerve injury. This means that SCO-Spondin derived peptides have been demonstrated able to remain in the blood circulation for a sufficient time, to cross blood barriers at a sufficient level to reduce effects of the injury, especially of spinal cord and/or optic nerve primary and/or secondary injury and help or improve recovery after such injury, especially after a spinal cord and/or optic nerve injury.

Systemic administration is unexpectedly efficient for the peptides of the invention in this particular context. It confers a significant advantage over administration directly at the lesion site or administration directly into the CSF (intrathecal or intraspinal injection) because systemic administration is safer and more convenient for the patient to be treated. An important or advantageous aspect of this mode of administration is that it renders possible repeated administrations by the health professional over time for a given patient. Another important or advantageous consequence of this way of administration is that it renders possible very early treatment, especially at the time of the emergency care process, and especially before the formation of a glial scar, and with an easy patient treatment acceptance. The peptides may be readily administered through injection or infusion, including via perfusion (the perfusion way being dedicated or not dedicated to the peptide, so that it is advantageously possible to use a perfusion used for the administration of other products). It is deemed that an early treatment may reduce or inhibit the toxicity resulting from initial cell death and the secondary injuries. The inventors also found that the bioavailability of these peptides at the lesion level after systemic administration does not require administration of unduly high amounts of peptide. This renders the systemic administration of the peptides very convenient and hopeful for a patient after such injury.

In a mouse model of focal lesion in the corpus callosum, systemic administration of peptides of the invention showed a beneficial effect on remyelination as shown with myelin binding protein level, Olig2-positive progenitor recruitment (required for synthesis of myelin sheath) and Olig2-positive cell generation. Systemic administration of SCO-Spondin derived peptides of the invention may thus be used for the remyelination in myelopathy, including spinal cord injury and the other forms of myelopathy related to the spinal cord or the CNS.

An object of the present invention is thus one or more peptide(s) derived from a Thrombospondin Repeat (TR or TSR) of SCO-spondin, or a pharmaceutical composition comprising one or more peptide(s) derived from a TR of SCO-spondin, for use in treating non-brain nervous system injury, such as spinal cord injury and/or optic nerve injury, wherein the peptide(s) is/are administered through a systemic route to the patient.

Another object of the present invention is one or more peptide(s) derived from a TR of SCO-spondin, or a pharmaceutical composition comprising one or more peptide(s) derived from a TR of SCO-spondin, for use in delivery of an efficient or sufficient amount of one or more of peptide(s) derived from a TR of SCO-spondin to injured non-brain nervous system, such as the spinal cord and/or to the optic nerve in a patient in need thereof, comprising administering said peptide(s) through a systemic route to the patient. This delivery is deemed to allow the peptide(s) to reach the cerebrospinal fluid. This delivery may be further characterized as allowing the delivered peptide(s) to treat spinal cord injury and/or optic nerve injury in a patient in need thereof.

Another object of the present invention is the use of one or more peptide(s) derived from a TR of SCO-spondin, for the manufacturing of a pharmaceutical composition for systemic administration to a patient for treating non-brain nervous system injury, such as spinal cord injury and/or optic nerve injury.

Another object of the present invention is a method of treatment of non-brain nervous system injury, such as spinal cord injury and/or optic nerve injury, in a patient in need thereof, comprising administering to said patient through a systemic route an efficient or sufficient amount of one or more peptide(s) derived from a TR of SCO-spondin, or of a pharmaceutical composition comprising the peptide(s).

Another object of the present invention is a method of delivery of an efficient or sufficient amount of one or more peptide(s) derived from a TR of SCO-spondin to injured non-brain nervous system, such as the spinal cord and/or to the optic nerve in a patient in need thereof, especially in the cerebrospinal fluid, comprising administering said peptide(s) to said patient through a systemic route. This delivery may be further characterized as allowing the delivered peptide(s) to treat spinal cord injury and/or optic nerve injury in a patient in need thereof.

In one aspect, this systemic administration of peptides of the invention has a beneficial effect on myelination or remyelination, as it may be evaluated, for example, using myelin binding protein measurement and/or Olig2-positive progenitor recruitment measurement and/or Olig2-positive cell generation measurement.

Another objet of the invention is one or more peptide(s) derived from a Thrombospondin Repeat (TR or TSR) of SCO-spondin, or a pharmaceutical composition comprising one or more peptide(s) derived from a TR of SCO-spondin, for use in remyelination in myelopathy, including spinal cord injury and the other forms of myelopathy related to the spinal cord or the CNS, wherein the peptide(s) is/are administered through a systemic route to the patient.

Another object of the present invention is a method of treatment of a myelopathy, or a method of remyelination, in a patient in need thereof, comprising administering to said patient through a systemic route an efficient or sufficient amount of one or more peptide(s) derived from a TR of SCO-spondin, or of a pharmaceutical composition comprising the peptide(s). The method of treatment of a myelopathy includes remyelination. The myelopathy includes spinal cord injury and the other forms of myelopathy related to the spinal cord or the CNS.

### DETAILED DESCRIPTION

“SCO-spondin” is a glycoprotein specific to the central nervous system and present in all of the vertebrates, from prochordals to humans. It is known as a molecule of extracellular matrices that is secreted by a specific organ located in the roof of the third ventricle, the sub-commissural organ. It is a molecule of large size. It consists of more than 4,500 amino acids and that has a multi-modular organization that comprises various preserved protein patterns, including in particular 26 TR or TSR patterns. It is known that certain peptides derived from SCO-spondin starting from TSR patterns have a biological activity in the nerve or neural cells (in particular described in WO-99/03890).

“TSR or TR patterns” are protein domains of approximately 55-60 residues, based on the alignment of preserved amino acids cysteine, tryptophan and arginine. These patterns were first isolated in TSP-1 (thrombospondin 1) that is a molecule that intervenes in coagulation. They were then described in numerous other molecules such as SCO-spondin. In fact, this thrombospondin type 1 unit (TSR) comprises, in all the proteins studied so far and previously mentioned, about 55-60 amino acids (AA) some of which, like cysteine (C), tryptophan (W), serine (S), glycine (G), arginine (R) and proline (P) are highly conserved.

“Systemic administration” means any mode of administration wherein a substantial part or a sufficient amount of the administered peptide(s) or peptide compound(s) reaches the blood circulation after such administration. Intrathecal administration is excluded as well as any systemic way of administration that does not target the peptide to blood circulation. The systemic administration route of the invention may be qualified of “blood-targeted systemic route of administration”. Further, in the context of the peptides or peptide compounds according to the invention, the peptides or peptide compounds once in the blood circulation are capable of passing through blood barriers, such as the BBB, the blood Spinal Cord barrier and/or the blood Optic nerve barrier.

Administration or use of “peptide” or “peptides” or “peptide(s), is a generic wording, and the invention encompasses administration or use of one single peptide or more than one single peptide, i.e. the administration or use of at least two peptides according to the present disclosure. Thus in the present disclosure the singular or the plural is not limited unless indicated to the contrary, and may each time encompass one single peptide, or at least two peptides. The same apply to the equivalent wording “peptide compound” that may be uses interchangeably for “peptide”.

“Spinal cord injury” means any damage notably caused by spinal section or compression. The spinal section can be caused by a trauma or by a surgery. The spinal compression can be caused by a trauma or secondary to the growth of surrounding cells such as in spinal tumor or spinal metastases. Spinal compression can also result from a disease affecting the spinal cord environment such as cervical arthrosic myelopathy or Schneider syndrome. The spinal compression can occur in the lumbar, thoracic and/or cervical region and the consequences of the injury for the patient will differ according to the location.

The “optic nerve” is a special sensory nerve that carries information from the visual world to the brain. Embryologically, the optic nerve is derived from an outgrowth of the forebrain; therefore, it is part of the central nervous system (CNS) and is composed of CNS fiber tracts. The article by Liang Li et al. (Frontiers in Cellular Neuroscience, April 2020, vol 14, article 109) explains that the mouse optic nerve crush (ONC) model has been widely used to study optic neuropathies and central nervous system (CNS) axon injury and repair. ONC provides a CNS neurodegeneration model that can be used for studying degenerative mechanisms and evaluating neuroprotectants and regeneration therapies. Conversely, results from a SCI or CNS injury model are deemed valuable for optic nerve injury.

“Optic nerve injury” means a condition which results from trauma or surgery or compression of optic nerve and can lead to vision reduction or loss.

“Injury” may be mild injury, moderate injury, or severe injury notably with partial or complete section of the spinal cord or of the optic nerve.

Myelopathy related to the spinal cord or the CNS includes in particular spinal cord injury, optic nerve injury, traumatic brain injury, multiple sclerosis, post-vaccination myelopathy, infectious myelopathy, viral myelopathy.

“Treating” or “treat” non-brain nervous system injury, such as spinal cord injury and/or optic nerve injury, means delivering an amount of peptide compound according to the invention and obtaining a favorable effect on the primary injury and/or secondary injury, in terms of inhibition of one or several detrimental effects of the injury, especially on the spinal cord and/or the optic nerve, such as: an inhibition or reduction of primary neural death and/or axonal degeneration; and/or reduction or inhibition of the toxicity resulting from primary cell death; and/or reduction or inhibition of the secondary consequences of the injury; and/or obtaining a benefice in terms of regeneration of the neural cells and/or of the axons; and/or obtaining a benefice in terms of functional recovery by the patient.

Description of the peptides or peptide compounds derived from a TR of SCO-spondin useful in performing the invention (the different objects of the invention, say peptide for use, method of use, method of treatment, use of a peptide for the manufacture of a medicament, etc.)

In particular, the invention uses a peptide of sequence

X1-W-S-A1-W-S-A2-C-S-A3-A4-C-G-X2 (SEQ ID NO: 1)

in which:

A1, A2, A3 and A4 consists of amino acid sequences consisting of 1 to 5 amino acids,

the two cysteines form a disulfide bridge or not,

X1 and X2 consists of amino acid sequences consisting of 1 to 6 amino acids; or X1 and X2 are absent;

it being possible for the N-terminal amino acid to be acetylated (e.g. bears H₃CCOHN—), for the C-terminal amino acid to be amidated (e.g. bears —CONH₂), or both the N-terminal amino acid to be acetylated and the C-terminal amino acid to be amidated.

In an embodiment, in the formula of SEQ ID NO: 1, X1 or X2 or both X1 and X2 are absent. In an embodiment, where X1 and/or X2 is absent, the N-terminal W is acetylated and/or the C-terminal G is amidated. Preferably, both X1 and X2 are absent and the N-terminal W is acetylated and the C-terminal G is amidated

In particular, the invention uses a peptide of sequence

W-S-A1-W-S-A2-C-S-A3-A4-C-G (SEQ ID NO: 2)

in which:
A1, A2, A3 and A4 consists of amino acid sequences consisting of 1 to 5 amino acids, the two cysteines form a disulfide bridge or not.

In an embodiment of the formulae of SEQ ID NO: 1 and 2, the peptide is a linear peptide, or the cysteines appearing on the peptide formula of SEQ ID NO: 1 and 2 do not form a disulfide bridge (reduced form).

In another embodiment, the two cysteines appearing on the peptide formula of SEQ ID NO: 1 and 2 form a disulfide bridge (oxidized form).

Preferably, in the formulae of SEQ ID NO: 1 and 2, A1, A2, A3 and/or, preferably and A4 consist preferably of 1 or 2 amino acids, more preferably of 1 amino acid.

Preferably, A1 is chosen from G, V, S, P and A, more preferably G, S.

Preferably A2 is chosen from G, V, S, P and A, more preferably G, S.

Preferably, A3 is chosen from R, A and V, more preferably R, V.

Preferably, A4 is chosen from S, T, P and A, more preferably S, T.

Preferably, A1 and A2 are independently chosen from G and S.

Preferably, A3-A4 is chosen from R-S or V-S or V-T or R-T.

When X1 is an amino acid sequence of 1 to 6 amino acids, the amino acids are any amino acid, and preferably chosen from V, L, A, P, and any combination thereof.

When X2 is an amino acid sequence of 1 to 6 amino acids, the amino acids are any amino acid, and preferably chosen from L, G, I, F, and any combination thereof.

In an embodiment, the peptide of SEQ ID NO: 1 or 2 is such that A1 and A2 are independently chosen from G and S and A3-A4 is chosen from R-S or V-S or V-T or R-T. In a particular modality, this peptide is further acetylated and/or amidated. In an embodiment, the peptide is a linear peptide, or the cysteines do not form a disulfide bridge. In another embodiment, the peptide have the two cysteines forming a disulfide bridge (C-terminal cyclization). In another embodiment, the peptide as used in the invention or the peptide administered to the patient through a systemic route does comprise both forms, oxidized peptide and linear peptide.

For the purposes of the present invention, the term “amino acids” means both natural amino acids and non-natural amino acids and changes of amino acids, including from natural to non-natural, may be made routinely by the skilled person while keeping the function or efficacy of the original peptide. By “natural amino acids” is meant the amino acids in L form that may be found in natural proteins, i.e. alanine, arginine, asparagine, aspartic acid, cysteine; glutamine, glutamic acid, glycine, histidine, isoleucine, leucine, lysine, methionine, phenylalanine, proline, serine, threonine, tryptophan, tyrosine and valine. By “non-natural amino acid” is meant the preceding amino acids in their D form, as well as the homo forms of certain amino acids such as arginine, lysine, phenylalanine and serine, or the nor forms of leucine or valine. This definition also comprises other amino acids such as alpha-aminobutyric acid, agmatine, alpha-aminoisobutyric acid, sarcosine, statin, ornithine, deaminotyrosine. The nomenclature used to describe the peptide sequences is the international nomenclature using the one-letter code and where the amino-terminal end is shown on the left and the carboxy-terminus is shown on the right. The dashes “-” represent common peptide bonds linking the amino acids of the sequences.

In an embodiment, the peptide according to the invention, for example any one of the peptides of sequence SEQ ID NO: 1-63, comprises an N-terminal acetylation, a C-terminal amidation, or both an N-terminal acetylation and a C-terminal amidation.

In different embodiments, the invention relates to the use of polypeptides consisting essentially of, or consisting of the following amino acid sequences (Table 1):

| | |
|---|---|
| W-S-G-W-S-S-C-S-R-S-C-G | SEQ ID NO: 3 |
| W-S-S-W-S-G-C-S-R-S-C-G | SEQ ID NO: 4 |
| W-S-S-W-G-S-C-S-R-S-C-G | SEQ ID NO: 5 |
| W-S-S-W-G-G-C-S-R-S-C-G | SEQ ID NO: 6 |
| W-S-S-W-S-S-C-S-R-S-C-G | SEQ ID NO: 7 |
| W-S-G-W-S-G-C-S-R-S-C-G | SEQ ID NO: 8 |
| W-S-G-W-G-S-C-S-R-S-C-G | SEQ ID NO: 9 |
| W-S-G-W-G-G-C-S-R-S-C-G | SEQ ID NO: 10 |
| W-S-S-W-S-S-C-S-V-S-C-G | SEQ ID NO: 11 |
| W-S-S-W-S-G-C-S-V-S-C-G | SEQ ID NO: 12 |
| W-S-S-W-G-S-C-S-V-S-C-G | SEQ ID NO: 13 |
| W-S-S-W-G-G-C-S-V-S-C-G | SEQ ID NO: 14 |
| W-S-G-W-S-S-C-S-V-S-C-G | SEQ ID NO: 15 |
| W-S-G-W-S-G-C-S-V-S-C-G | SEQ ID NO: 16 |
| W-S-G-W-G-S-C-S-V-S-C-G | SEQ ID NO: 17 |
| W-S-G-W-G-G-C-S-V-S-C-G | SEQ ID NO: 18 |
| W-S-S-W-S-S-C-S-V-T-C-G | SEQ ID NO: 19 |
| W-S-S-W-S-G-C-S-V-T-C-G | SEQ ID NO: 20 |
| W-S-S-W-G-S-C-S-V-T-C-G | SEQ ID NO: 21 |
| W-S-S-W-G-G-C-S-V-T-C-G | SEQ ID NO: 22 |
| W-S-G-W-S-S-C-S-V-T-C-G | SEQ ID NO: 23 |
| W-S-G-W-S-G-C-S-V-T-C-G | SEQ ID NO: 24 |
| W-S-G-W-G-S-C-S-V-T-C-G | SEQ ID NO: 25 |
| W-S-G-W-G-G-C-S-V-T-C-G | SEQ ID NO: 26 |
| W-S-S-W-S-S-C-S-R-T-C-G | SEQ ID NO: 27 |
| W-S-S-W-S-G-C-S-R-T-C-G | SEQ ID NO: 28 |
| W-S-S-W-G-S-C-S-R-T-C-G | SEQ ID NO: 29 |
| W-S-S-W-G-G-C-S-R-T-C-G | SEQ ID NO: 30 |
| W-S-G-W-S-S-C-S-R-T-C-G | SEQ ID NO: 31 |
| W-S-G-W-S-G-C-S-R-T-C-G | SEQ ID NO: 32 |
| W-S-G-W-G-S-C-S-R-T-C-G | SEQ ID NO: 33 |
| W-S-G-W-G-G-C-S-R-T-C-G | SEQ ID NO: 34 |

In an embodiment, the peptides of sequences SEQ ID NO: 3-34 disclosed in Table 1 are linear peptides, or the cysteines do not form a disulfide bridge (reduced peptides). In another embodiment, the peptides of sequences SEQ ID NO: 3-34 disclosed in the preceding table have the two cysteines oxidized to form a disulfide bridge (oxidized peptides). In another embodiment, the peptides as used in the invention or the peptides administered to the patient through a systemic route do comprise both forms, oxidized peptide and linear peptide of the same peptide sequence. In still another embodiment, the peptides as used in the invention or the peptides administered to the patient through a systemic route does comprise a mixture of at least two of these different peptides chosen from sequences SEQ ID NO: 3-34, wherein the mixture may be a mixture of at least two linear peptides or a mixture of at least two oxidized peptides, or a mixture of at least one linear peptide and at least one oxidized peptide, for example having the same amino acid sequence.

In a preferred embodiment, the peptide consists of the amino acid sequence W-S-G-W-S-S-C-S-R-S-C-G (SEQ ID NO: 3). In an embodiment, the peptide is a linear peptide, or the cysteines do not form a disulfide bridge (reduced form). In another embodiment, the peptides have the two cysteines oxidized to form a disulfide bridge (oxidized form). In another embodiment, the peptides as used in the invention or the peptides administered to the patient through a systemic route does comprise both forms, oxidized and reduced.

In an embodiment of the peptides of SEQ ID NO: 1,
X1 represents a hydrogen atom or P or A-P or L-A-P or V-L-A-P, and/or
X2 represents a hydrogen atom or L or L-G or L-G-L or L-G-L-I or L-G-L-I-F.

In different embodiments, the invention thus relates to the use of polypeptides consisting or consisting essentially of the following amino acid sequences (Table 2):

| | |
|---|---|
| P-W-S-G-W-S-S-C-S-R-S-C-G | SEQ ID NO: 35 |
| A-P-W-S-G-W-S-S-C-S-R-S-C-G | SEQ ID NO: 36 |
| L-A-P-W-S-G-W-S-S-C-S-R-S-C-G | SEQ ID NO: 37 |
| V-L-A-P-W-S-G-W-S-S-C-S-R-S-C-G | SEQ ID NO: 38 |
| W-S-G-W-S-S-C-S-R-S-C-G-L | SEQ ID NO: 39 |
| W-S-G-W-S-S-C-S-R-S-C-G-L-G | SEQ ID NO: 40 |
| W-S-G-W-S-S-C-S-R-S-C-G-L-G-L | SEQ ID NO:41 |
| W-S-G-W-S-S-C-S-R-S-C-G-L-G-L-I | SEQ ID NO: 42 |
| W-S-G-W-S-S-C-S-R-S-C-G-L-G-L-I-F | SEQ ID NO: 43 |
| P-W-S-G-W-S-S-C-S-R-S-C-G-L | SEQ ID NO:44 |
| P-W-S-G-W-S-S-C-S-R-S-C-G-L-G | SEQ ID NO: 45 |
| P-W-S-G-W-S-S-C-S-R-S-C-G-L-G-L | SEQ ID NO: 46 |
| P-W-S-G-W-S-S-C-S-R-S-C-G-L-G-L-I | SEQ ID NO: 47 |
| P-W-S-G-W-S-S-C-S-R-S-C-G-L-G-L-I-F | SEQ ID NO: 48 |
| A-P-W-S-G-W-S-S-C-S-R-S-C-G-L | SEQ ID NO: 49 |
| A-P-W-S-G-W-S-S-C-S-R-S-C-G-L-G | SEQ ID NO: 50 |
| A-P-W-S-G-W-S-S-C-S-R-S-C-G-L-G-L | SEQ ID NO: 51 |
| A-P-W-S-G-W-S-S-C-S-R-S-C-G-L-G-L-I | SEQ ID NO: 52 |
| A-P-W-S-G-W-S-S-C-S-R-S-C-G-L-G-L-l-F | SEQ ID NO: 53 |
| L-A-P-W-S-G-W-S-S-C-S-R-S-C-G-L | SEQ ID NO: 54 |
| L-A-P-W-S-G-W-S-S-C-S-R-S-C-G-L-G | SEQ ID NO: 55 |
| L-A-P-W-S-G-W-S-S-C-S-R-S-C-G-L-G-L | SEQ ID NO: 56 |
| L-A-P-W-S-G-W-S-S-C-S-R-S-C-G-L-G-L-I | SEQ ID NO: 57 |
| L-A-P-W-S-G-W-S-S-C-S-R-S-C-G-L-G-L-I-F | SEQ ID NO: 58 |
| V-L-A-P-W-S-G-W-S-S-C-S-R-S-C-G-L | SEQ ID NO: 59 |
| V-L-A-P-W-S-G-W-S-S-C-S-R-S-C-G-L-G | SEQ ID NO: 60 |
| V-L-A-P-W-S-G-W-S-S-C-S-R-S-C-G-L-G-L | SEQ ID NO: 61 |
| V-L-A-P-W-S-G-W-S-S-C-S-R-S-C-G-L-G-L-I | SEQ ID NO: 62 |
| V-L-A-P-W-S-G-W-S-S-C-S-R-S-C-G-L-G-L-I-F | SEQ ID NO: 63 |

In an embodiment, the peptides of sequences SEQ ID NO: 35-63 disclosed in Table 2, or of sequences SEQ ID NO: 3-63 disclosed in Tables 1+2, are linear peptides, or the cysteines do not form a disulfide bridge (reduced peptides). In another embodiment, the peptides have the two cysteines oxidized to form a disulfide bridge (oxidized peptides). In another embodiment, the peptides as used in the invention or the peptides administered to the patient through a systemic route does comprise both forms, oxidized peptide and linear peptide of the same peptide sequence. In still another embodiment, the peptides as used in the invention or the peptides administered to the patient through a systemic route does comprise a mixture of at least two of these different peptides chosen from sequences SEQ ID NO: 35-63, or 3-63, wherein the mixture may be a mixture of at least two linear peptides or a mixture of at least two oxidized peptides, or a mixture of at least one linear peptide and at least one oxidized peptide, for example having the same amino acid sequence.

Each one of the peptides of sequences SEQ ID NO: 3-63 may be acetylated, amidated, or acetylated and amidated.

In the present invention, the peptides as used in the invention or the peptides administered to the patient through a systemic route are defined with their amino acid sequences. The peptides as used may be one peptide as disclosed herein, or a mixture of at least two peptides as disclosed herein. The mixtures also encompass the mixture of linear and oxidized peptides, of the same or different amino acid sequences. If a 100% pure peptide may be used, in accordance with the invention, it is possible, and the invention encompasses, that the peptide has a purity greater than 80%, preferably 85%, more preferably 90%, even more preferably equal to or greater than 95, 96, 97, 98, or 99%. Conventional purification methods, for example by chromatography, may be used to purify the desired peptide compound.

In an embodiment, the peptide as used in the invention or the peptide administered to the patient through a systemic route does comprise both forms, oxidized peptide (Op) and linear peptide (Lp), for instance in similar amounts or not, e.g. (% in number) Op: 10, 20, 25, 30, 40, 50, 60, 70, 80, or 90%, the remaining to 100% being the Lp. The oxidized peptide and the linear peptide that are combined may be of the same sequence or of different sequences. For example, the oxidized and linear forms of the peptide of sequence SEQ ID NO: 3 are so combined. The same apply to any one of the peptides of sequence SEQ ID NO: 4-34 and 35-63.

The pharmaceutical composition as used in the invention comprises as active ingredient a peptide or mixture of peptides as previously described, for example peptides of different amino acid composition or peptides of the same amino acid composition under oxidized and linear forms, and one or more pharmaceutically-acceptable vehicles, carriers or excipients.

The peptide compounds according to the invention may be used in a pharmaceutical composition or in the manufacture of a medicament. In these compositions or medicaments, the active principle may be incorporated into compositions in various forms, i.e. in the form of solutions, generally aqueous solutions, or in freeze-dried form, or in the form of emulsion or any other pharmaceutically and physiologically acceptable form suited to systemic administration route.

In accordance with an important feature of the invention, the peptide compound or the composition containing the same is administered through a systemic route. Mention may be made in particular of the following injection or administration routes: intravenous, intraperitoneal, intranasal, subcutaneous, intramuscular, sublingual, oral, and combinations thereof.

In accordance with the invention, administration may be performed at different timepoints after the injury or the suspicion of injury.

In an embodiment, the administration is performed in time close to the spinal cord injury and/or optic nerve injury occurrence or close to the accident or surgery and this includes a suspicion of spinal cord injury and/or optic nerve injury. Systemic administration routes indeed allow a first administration or the beginning of a treatment very early in time, especially as soon as the medical aid is present and there is a diagnosis or a suspicion of spinal cord and/or optic nerve injury. The administration may begin at the accident location or in the ambulance, helicopter and the like, or at the surgical room or at the hospital, clinic and the like.

In accordance with the invention, when the injury is a trauma caused by an internal source, such as a tumor, administration may be performed as soon as the trauma is suspected, is observed or is prognosticated to occur. In an embodiment, if there is a surgery made to eliminate all or part of the tumor, administration may be performed before or after or concomitant to surgery, as described above.

The treatment may be performed or may begin in the first days (e.g. the same day or within a week), or several weeks (e.g. 1-8) or months (e.g. 2-6) after the injury.

In an embodiment, the treatment or the first administration is made early, e.g. 10 minutes, 30 minutes, 1, 2, 3, 4, 5 hour(s) after the occurred injury, the suspected injury or the prognostic is made.

In another embodiment, the treatment or the first administration is made within a period of 12, 24, 36 or 48 hours after the occurred injury, the suspected injury or the prognostic is made.

In an embodiment, the treatment according to the invention is made to a patient that has been treated, is being treated or will be treated with a medicament inhibiting or reducing gliosis.

One dose expressed in weight of peptide per patient body weight (kg) may range from about 1 μg/kg to about 1 g/kg, in particular from about 10 μg/kg to about 100 mg/kg, e.g. from about 50 μg/kg to about 50 mg/kg.

The dosage regimen may comprise a single administration or repeated administrations. According to an embodiment, repeated administrations may comprise administering one dose per day of treatment, for example one dose every day or every 2 or 3 days over a treatment period. According to another embodiment, repeated administrations may comprise administering at least two doses per day of treatment, for example 2, 3 or more doses per day over a treatment period. In these embodiments, a treatment period may be 1, 2, 3, 4, 5, 6, 7, 8, 9, 10 or more days.

In an embodiment a dose is administered by perfusion. Perfusion may last several minutes, tens of minutes, hours, and up to 24 hours a day.

The use according to the invention and the method of treatment of the invention may be characterized as allowing delivery of an amount of peptide compound according to the invention and obtaining a favorable effect on the primary and/or secondary injury, in terms of inhibition of one or several detrimental effects of the injury, especially on the spinal cord and/or the optic nerve. This favorable effect may comprise:
an inhibition or reduction of primary neural cell death and/or axonal degeneration;
an inhibition or reduction of primary neuronal cell death and/or axonal degeneration;
a reduction or inhibition of the toxicity resulting from primary cell death;
a reduction or inhibition of the secondary consequences of the injury;
obtaining a benefice in terms of regeneration of the neuronal cells and/or of the axons and/or of the myelin sheath;
obtaining a benefice in terms of functional recovery by the patient.

In an embodiment, the use or the method of treatment has the effect of inhibiting or reducing neural cell death and/or axonal degeneration, and/or necrosis (primary injury), secondary injuries (especially inhibition or reduction of secondary neural cell death and/or axonal degeneration).

In an embodiment, the use or the method of treatment has the effect of inducing or favoring neural path recovery or regeneration.

In an embodiment, the use or the method of treatment has the effect of helping or inducing functional recuperation, meaning that the patient recovers all or part of the functions lost as the result of the injury.

In an embodiment, the use or the method of treatment has the effect of stopping or inhibiting functional loss resulting from injury.

The invention also relates to one or more peptide(s) as described herein or a pharmaceutical composition as described herein, for use in remyelination in a myelopathy, wherein the peptide(s) is/are administered through a systemic route to the patient.

The invention also relates to a method of treatment of a myelopathy, or a method of remyelination, in a patient in need thereof, comprising administering to said patient through a systemic route an efficient or sufficient amount of one or more peptide(s) as described herein or a pharmaceutical composition as described herein. The method of treatment of a myelopathy includes remyelination.

In an embodiment, the myelopathy is spinal cord injury or optic nerve injury. In another embodiment, the myelopathy is another form of myelopathy related to the spinal cord or the CNS, including in particular traumatic brain injury, multiple sclerosis, post-vaccination myelopathy, infectious myelopathy, viral myelopathy

Dose regimens, administration routes, selection of peptides, and any useful features as disclosed above are applicable to these two last objects.

The invention also relates to the use of these peptides or a method of treatment using these peptides, in order to induce myelination of neurons, in vitro or in vivo.

In one aspect, this systemic administration of peptides of the invention has a beneficial effect on myelination or remyelination. These effects may be measured using known methods and in particular using a method allowing measuring the level of myelin binding protein (MBP) at a lesion site in a patient or an animal model, and/or measuring the level of Olig2-positive progenitor recruitment and Olig2-positive cells generation at a lesion site in a patient or an animal model.

In an embodiment, measuring the level of myelin binding protein (MBP) at a lesion site in a patient or an animal model may be made using MBP immunostaining with antibodies (see example 4 for a method and for an antibody). It is possible to use a control with vehicle and no peptide according to the invention, or to compare with predetermined values.

In another embodiment, measuring the level of Olig2-positive progenitor recruitment and Olig2-positive cells generation at a lesion site in a patient or an animal model, may be made using measuring the Olig2 cell amount or density, e.g. with antibodies (see example 4 for a method and for an antibody). It is possible to use a control with vehicle and no peptide according to the invention, or to compare with predetermined values.

In another embodiment, both measuring are made.

The invention also proposes to combine treatment with the peptides and measurement of the remyelination, based on those measuring methods.

The present invention will now be described in more details using non-limiting examples referring to the figures:

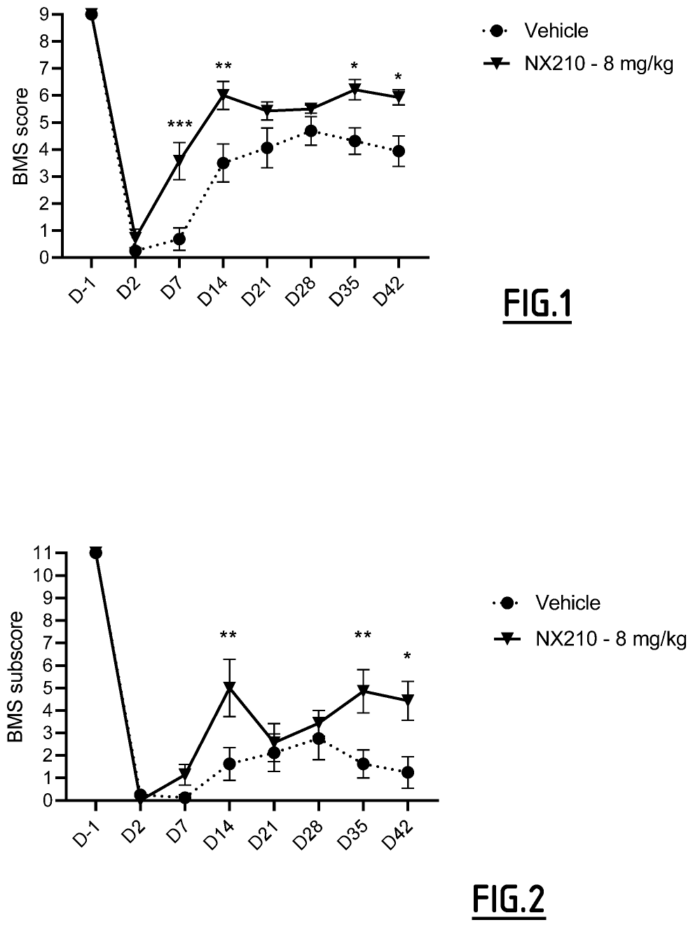

FIG. 1: Assessing recovery of hindlimb function after a T8 dorsal hemisection (spinal cord injury) using open field locomotion test, giving the BMS score in vehicle treated mice (n=8) and NX210 treated mice (n=7). *: p<0.05, **: p<0.01, ***: p<0.001. Two-way ANOVA followed by Bonferroni's post hoc test.

FIG. 2: Assessing recovery of hindlimb function after a T8 dorsal hemisection (spinal cord injury) using open field locomotion test, giving the BMS subscore in vehicle treated mice (n=8) and NX210 treated mice (n=7). *: p<0.05, **: p<0.01. Two-way ANOVA followed by Bonferroni's post hoc test.

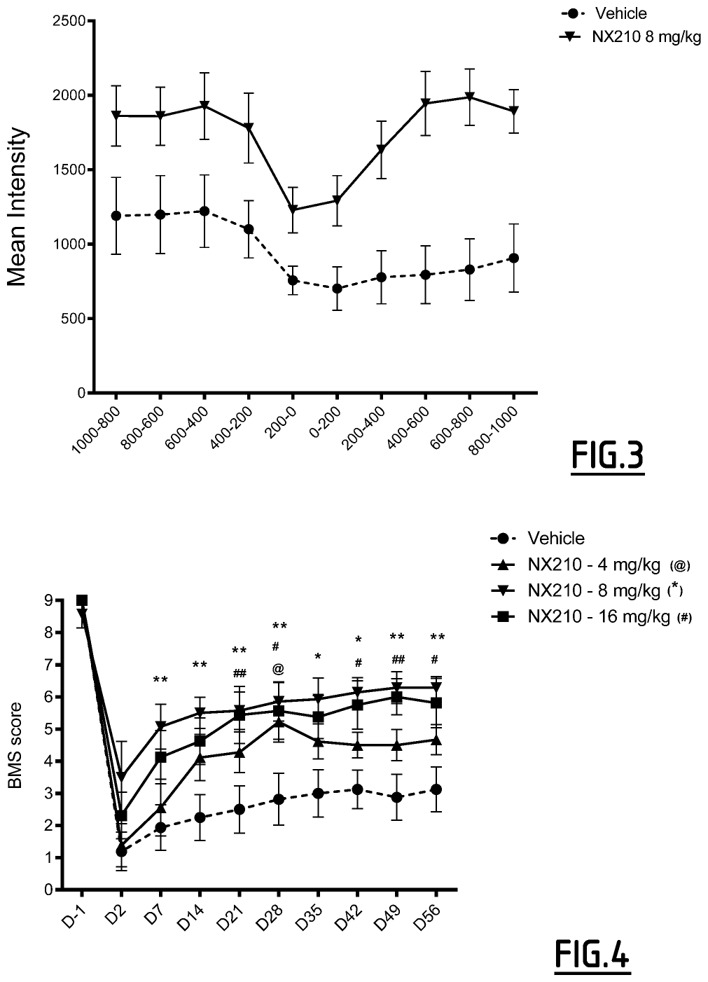

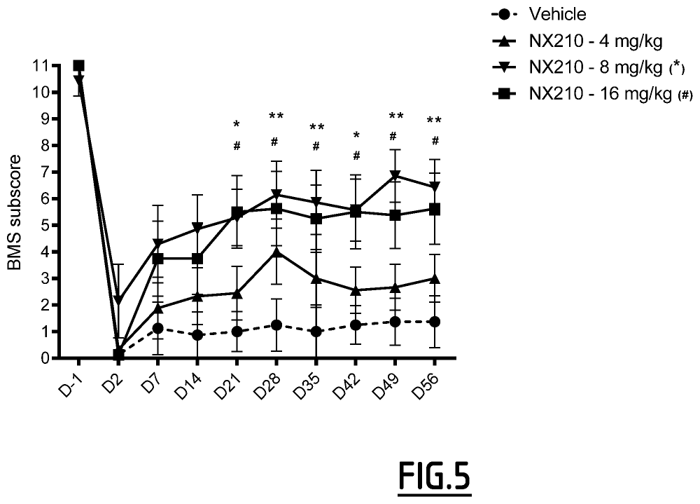

FIG. 3: Assessing myelin basic protein (MBP) staining intensity after a T8 dorsal hemisection (spinal cord injury) 1000 μm rostral and 1000 μm caudal to the injury using immunofluorescence in vehicle treated mice (n=3) and NX210 treated mice (n=5). FIG. 4: Assessing recovery of hindlimb function after a T8 dorsal hemisection (spinal cord injury) using open field locomotion test, giving the BMS score in vehicle treated mice (n=8) and mice treated with 4 mg/kg (n=9), 8 mg/kg (n=7) and 16 mg/kg (n=8). @ or * or #: p<0.05, ** or ##: p<0.01. Two-way ANOVA followed by Bonferroni's post hoc test.

FIG. 5: Assessing recovery of hindlimb function after a T8 dorsal hemisection (spinal cord injury) using open field locomotion test, giving the BMS subscore in vehicle treated mice (n=8) and mice treated with 4 mg/kg (n=9), 8 mg/kg (n=7) and 16 mg/kg (n=8). * or #: p<0.05, **: p<0.01. Two-way ANOVA followed by Bonferroni's post hoc test.

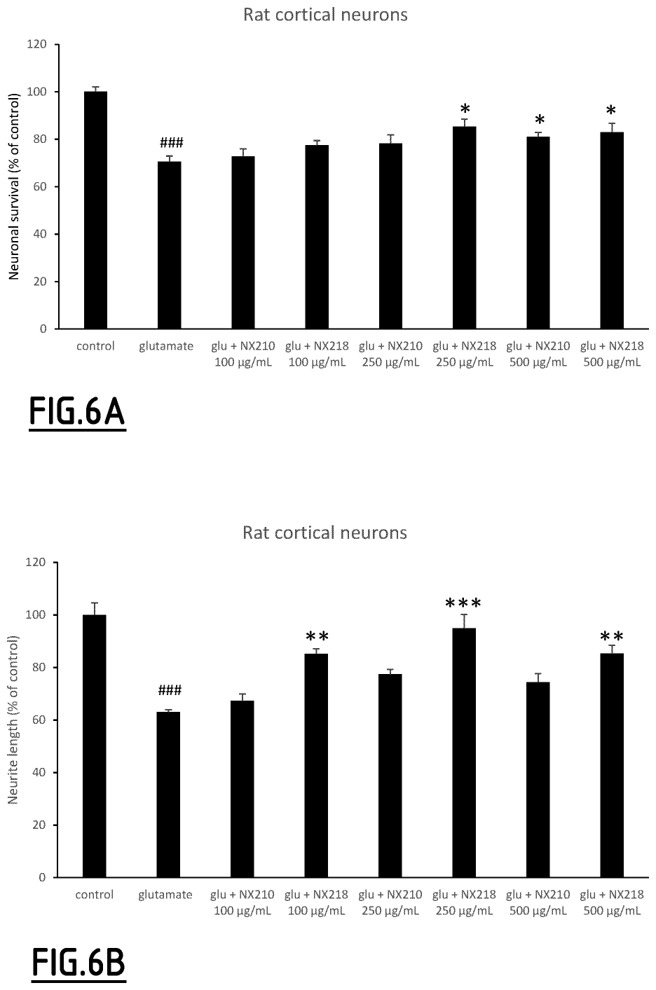

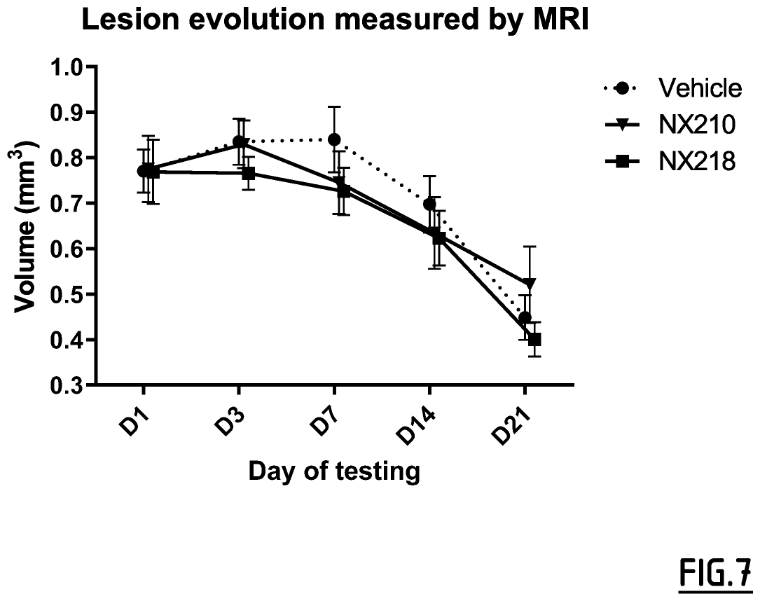

FIG. 6: NX210 and NX218 (NX210 oxidized form) peptides protected rat cortical neurons against glutamate-induced excitotoxicity in vitro. Primary cortical neurons isolated from E15 rat embryos were co-treated with glutamate (glu, 20 μM), and either vehicle, NX210 or NX218 (100, 250, 500 μg/mL) for 20 minutes at 13 days in vitro. Two days later, neurons were fixed and immunostained with the neuronal marker microtubule associated protein-2 (MAP-2). A. Neuronal survival was assessed by the number of MAP-2-positive neurons. B. Neurite network was assessed by the cumulative neurite length of MAP-2-positive neurons. A,B. Data are presented as median and interquartile range. Kruskal-Wallis followed by Dunn's test: ###p<0.001 control vs glu; ***p<0.001, **p<0.01, *p<0.05 glu vs glu+NX; $$p<0.01, $p<0.05 NX210 vs NX218, n=5-6.

FIG. 7: Lesion volume follow-up after lysolecithin injection using Magnetic Resonance Imagery (MRI) in vehicle-treated mice (n=7) and mice treated with 5 mg/kg of either NX210 (n=8) or NX218 (n=8). Two-way ANOVA followed by Bonferroni's post hoc test.

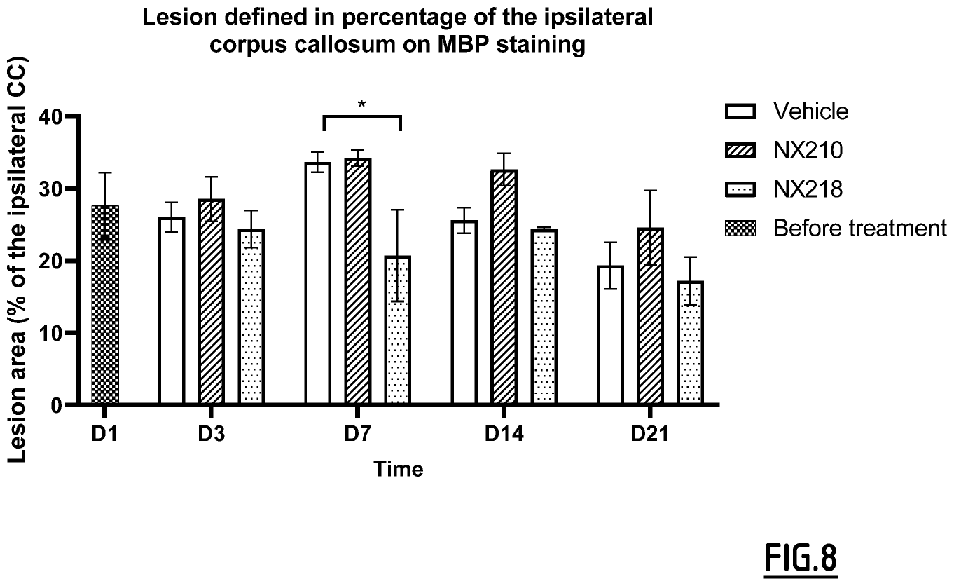

FIG. 8: Assessing lesion area after lysolecithin injection using Myelin Binding Protein (MBP) immunostaining in vehicle-treated mice (n=3 per timepoint) and mice treated with 5 mg/kg of either NX210 (n=3 per timepoint) or NX218 (n=3 per timepoint). Lesion area expressed as a percentage of the ipsilateral corpus callosum (CC). *: p<0.05, t-test.

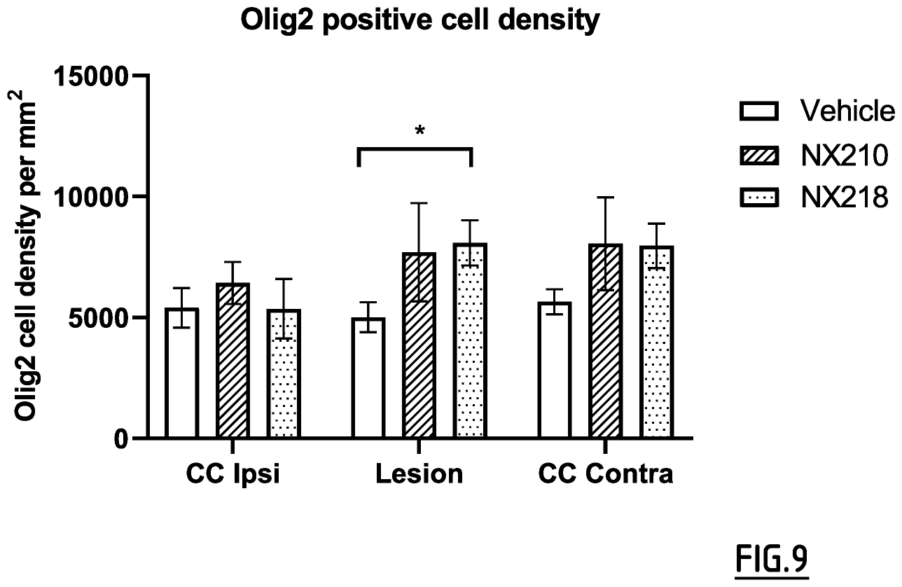

FIG. 9: Assessing the Olig2-positive cell density in the corpus callosum (CC) after lysolecithin injection using Olig2 immunostaining in vehicle-treated mice (n=3 per timepoint) and mice treated with 5 mg/kg of either NX210 (n=3 per timepoint) or NX218 (n=3 per timepoint). *: p<0.05, t-test.

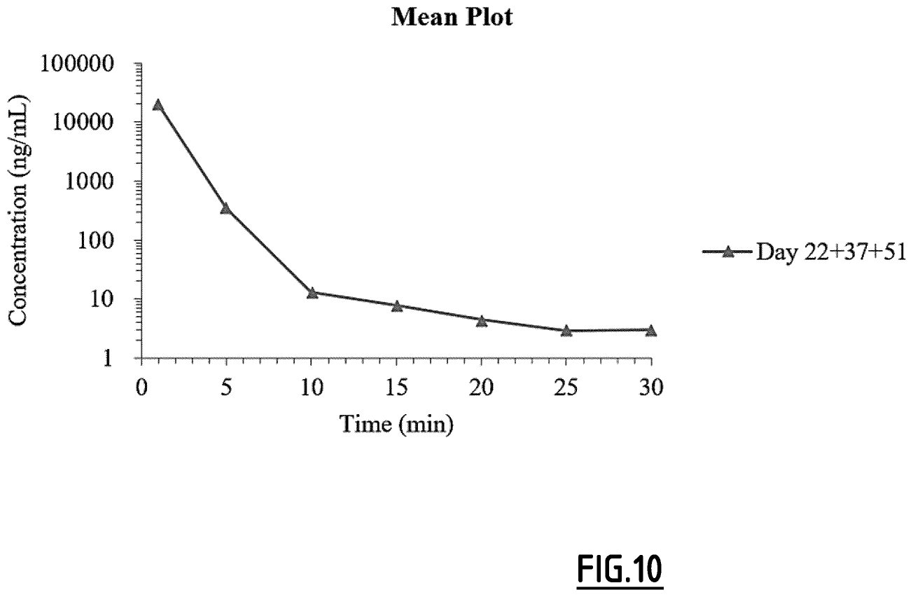

FIG. 10: PK profile after NX210 IV administration in Monkey (bolus IV injection of 10 mg/kg of NX 210). Mean monkey plasma concentration (ng/mL) of NX218 (NX210 cyclic form) is plotted on y-axis with decimal logarithmic scale.

### EXAMPLE 1: SYNTHESIS OF NX PEPTIDES

The manufacturing process of the peptides of sequence SEQ ID NO: 1, 2, or of any of the sequences 3-63, and especially those used in the Part Example, such as NX210 (SEQ ID NO: 3), is based on Solid-Phase Peptide Synthesis applying N-α-Fmoc (side chain) protected amino acids as building blocks in the assembly of the peptide. The protocol employed consists of a coupling of the C-terminal Glycine N-α-Fmoc-protected amino acid bound to an MPPA linker on the MBHA resin, followed by Fmoc coupling/deprotection sequences. After assembly of the peptide on the resin, a step of simultaneous cleavage of the peptide from the resin and deprotection of the side chains of amino acid is carried-out.

The crude peptide is precipitated, filtered and dried. Prior to purification by preparative reverse phase chromatography, the peptide is dissolved in an aqueous solution containing acetonitrile. The purified peptide in solution is the concentrated before undergoing an ion exchange step to obtain the peptide in the form of its acetate salt.

The skilled person may refer for further detail of synthesis to U.S. Pat. No. 6,995,140 and WO2018146283, and for the oxidized forms of the peptides disclosed herein, to WO 2017/051135, all incorporated herein by reference.

The skilled person further has access to the standard methods to produce any of the disclosed peptides of the invention including the N-ter and C-ter modified or protected peptides. Concerning the acetylation and/or the amidation of the peptides at the N-terminal and C-terminal respectively, the skilled person may refer to standard techniques, e.g. those described in Biophysical Journal Volume 95 November 2008 4879-4889, also incorporated by reference.

### EXAMPLE 2: FUNCTIONAL RECOVERY OF MICE TREATED WITH NX210 AFTER DORSAL HEMISECTION (SPINAL CORD INJURY)

### Material & Methods

### Drug Administration

SCO-Spondin derived peptide (NX210) was solubilized in its vehicle (water for injection). Administrations of NX210 peptide were performed via intraperitoneal (i.p.) route starting at D0, either:
Ten minutes post injury and repeated twice a week for 7 weeks at 8 mg/kg.
Four hours post injury and repeated twice a week for 10 weeks at 4, 8 or 16 mg/kg.

### Surgical Procedures

### Animals

Female C57Bl/6 mice, 6-8 weeks old and weighing ˜18-20 g were kept for housing in groups with access to food and water ad libitum. They were kept in a temperature and humidity-controlled animal facility (temperature 22° C., relative humidity 52%) on a 12 h/12 h light/dark cycle. Mice were numbered with ear tags.

### Spinal Cord T8 Dorsal Hemisection:

The dura was punctured bilaterally with a 30G needle at appropriate locations (Geoffroy CG. Et al. J Neurosci. 2015 Apr. 22; 35(16):6413-28). Then, a pair of superfine iridectomy scissors was used to cut the spinal cord: the dorsal half of the cord at a depth of 0.8 mm for dorsal hemisection. Finally, a micro feather ophthalmic scalpel was used to retrace the lesion to ensure its completeness. The muscles were sutured with 5.0 sutures and the skin was secured with 5.0 sutures and Dermabond was used to glue the skin.

### Randomization of the Animals

In each cage (maximum n=5 per cage), mice were randomly distributed by a non-observer. Surgeon were given syringes anonymously labeled, with content unknown to him. Animals were tested in a random and double-blind manner: all behavioral tests were performed by observers blind to the drug treatment and quantified by different observers also blind to the drug treatment groups.

### Mortality/Observation of Animals

Health of animals were checked daily. General aspect of animals and their activity were monitored daily whereas their weight was monitored once every week. Acute or delayed mortality were checked.

### Behavioral Tests

### Open Field—BMS

For the BMS score (Basso Mouse Scale, adaptation of the widely used BBB scoring system for rats, Basso DM. et al. J Neurotrauma. 2006 May, 23(5):635-59,), mice were placed in an open field and observed for 5 minutes by two observers blinded to treatment (Geoffroy et al., 2015). Many features were noted, including ankle movements, stepping pattern, frequency, coordination, paw placement, trunk instability and tail position. The BMS scores were calculated, ranging from 0 (no movement) to 9 (normal locomotion). Mice were tested weekly for open field-BMS.

### Rotarod

For rotarod tests, mice were placed on a rod (Ugo Basile) rotating at increasing speeds from 5 rpm to 50 rpm during 3 minutes with constant acceleration. The latency to fall (in seconds) was averaged with two trials per session. One week before injury mice were first acclimated to the test for five days (2 sessions) with an additional session one day prior injury (baseline). Mice were tested weekly for rotarod assay (Geoffroy et al., 2015).

### Activity Chamber

Locomotor activity was recorded by placing the mice in an open-field arena equipped with light beams arrays in the horizontal X and Y axes. The hardware detects beam paths broken by the animal and determine the location of the rodent within the cage. This chamber provides information regarding overall activity of the mice in the chamber (e.g. total number of movements). Mice were trained twice before testing, then tested at day ˜1 and then weekly. Mice activity was recorded for 10 min during each session.

### Euthanasia and Tissue Sampling

On day 56 (experiment 1) or on day 73 (experiment 2), animals were sacrificed. Mice were given lethal dose of Fatal plus (pentobarbital), and perfused transcardially with PBS-Heparin (10,000 unit/L, 20 ml, 5 ml/min) followed by 4% paraformaldehyde (30-40 ml/mouse, at 5 ml/min). After removal of the spinal cord, the tissues were post fixed overnight at 4° C. in the same fixative solution. Tissue was incubated in 30% sucrose for 3 days for cryo-protection. Different segments of the spinal cord (4 mm rostral and 4 mm caudal to the injury for the dorsal hemisection group, as well as a segment 4 mm above injury) were embedded in OCT compound, and snapped frozen on dry ice. Spinal cords were sectioned with a cryostat (Thermofisher, Microm HM550) at 25 μm thickness for further processing and stored at −20° C. in a cryoprotectant solution (Sucrose 30%, Ethylene Glycol, PBS). All the tissue processing, staining and quantification were performed by observers blind to the group treatment.

### Analyses

### Immunohistochemistry

Floating consecutive serial sagittal sections centered at the injury site were stained for Myelin Basic Protein (MBP) using monoclonal rat anti-MBP (overnight incubation, MAB386 Millipore) after blocking in PBS-Tx-0.4% with 5% Horse Serum for 2 hours at room temperature. The next day, sections were washed and secondary anti-rat Alexa fluor 488 (1:500, A-11006, Thermofisher) was added for 1 hour at room temperature. After several washes with PBS, all sections were stained with DAPI prior to cover-slipping with Fluoromount-G (Southern Biotech).

### Immunoreactivity Measurement

The lesion severity was determined by measuring the injury size, determined by the MBP negative area, and the maximum depth of the injury. Effect on myelin sheath was assessed by measuring the MBP staining intensity at different distance rostral and caudal to injury. After MBP immunostaining, a series of rectangles of 100 μm wide covering the entire dorsoventral axis of the spinal cord was superimposed onto sagittal sections, starting from the injury site all the way to 1.0 mm rostral to the injury. After subtraction of the background, intensity for MBP was measured in every rectangle using ImageJ and normalized against the intensity at 1.0 mm away to the injury. The ratio was taken as the Staining Intensity Ratio and was plotted as function of the distance to the injury. Three spinal cord sections were averaged for each animal.

### Statistical Analysis

All values were expressed as mean±S.E.M. Statistic analyses were performed on the different conditions using a two-way ANOVA followed by Bonferroni's post hoc test for behavior. Values of p<0.05 were considered as statistically significant.

### Results

### Model of Spinal Cord Injury—T8 Dorsal Hemisection—Experiment 1

After a T8 dorsal hemisection in mice, NX210 peptide (8 mg/kg) was administered twice a week via intraperitoneal (i.p.) route, the first injection being performed 10 minutes post-injury. The locomotor function in NX210- or vehicle-treated mice were assessed weekly using the Basso Mouse Scale (BMS) open field test (Tables 1, 2 & 3—FIGS. 1 & 2) and the Rotarod test to analyze the performance under forced movement (Table 4). Post-mortem analyses were also performed using immunostainings (notably MBP labelling, Table 5 & FIG. 3).

Analysis of locomotor activity using the BMS open field test revealed a significant increased recovery of NX210-treated mice when compared to vehicle-injected mice (Table 1 & FIG. 1). BMS score was significantly higher in the NX210-treated mice from day 7 to the end of the study (day 42 post-injury).

A similar result was obtained in the BMS subscore (Table 2 & FIG. 2), which can detect differences in finer details of locomotion that may not be apparent in the overall BMS score.

The percentage of mice reaching a BMS score superior or equal to 5 corresponds to plantar stepping and some coordination. Interestingly, all NX210-treated mice (100%) had a BMS score superior or equal to 5 at the end of the study (at day 42 post-injury) demonstrating functional recovery, compared to only 37.5% of vehicle-treated mice (Table 3).

Analysis of locomotor activity using the rotarod test demonstrated that NX210 administrations increased the duration time on the rod and latency to fall (Table 4).

Spinal Cord Injury, notably during the secondary injury stage, induces strong demyelination throughout the rostrocaudal axis of the lesion. Eight weeks after injury, spinal cord sections (1000 μm rostral and 1000 μm caudal to the injury) were stained with Myelin Basic Protein (MBP) which detects myelin protein and myelin sheath. After quantification, MBP immunostaining revealed significant increase in MBP intensity levels both at the lesion site and throughout the 1000 μm rostral and caudal distances, in NX210-treated mice compared to vehicle treated mice (Table 5 and FIG. 3).

Taken together, these locomotor and histological data highlighted the improvement of functional and biological recovery, and demonstrated the benefits from administrations of NX210, an SCO-Spondin derived peptide, after Spinal Cord Injury.

TABLE 1
Assessing recovery of hindlimb function after a T8 dorsal
hemisection (spinal cord injury) using open field locomotion
test, giving the BMS score in vehicle treated mice
(n = 8) and NX210 treated mice (n = 7).

| | | | | NX210 | | |
|---|---|---|---|---|---|---|
| | Vehicle | | | 8 mg/kg | | |
| | BMS score | | | BMS score | | |
| | Mean | SEM | n | Mean | SEM | n |
| D-1 | 9.00 | 0.00 | 8 | 9.00 | 0.00 | 7 |
| D 2 | 0.25 | 0.13 | 8 | 0.71 | 0.34 | 7 |
| D 7 | 0.69 | 0.41 | 8 | 3.57*** | 0.69 | 7 |
| D 14 | 3.50 | 0.70 | 8 | 6.00** | 0.52 | 7 |
| D 21 | 4.06 | 0.73 | 8 | 5.43 | 0.34 | 7 |
| D 28 | 4.69 | 0.53 | 8 | 5.50 | 0.15 | 7 |
| D 35 | 4.31 | 0.49 | 8 | 6.21* | 0.38 | 7 |
| D 42 | 3.94 | 0.56 | 8 | 5.93* | 0.28 | 7 |

*p < 0.05,
**p < 0.01,
***p < 0.001.
Two-way ANOVA followed by Bonferroni's post hoc test.

TABLE 2
Assessing recovery of hindlimb function after a T8 dorsal
hemisection (spinal cord injury) using open field locomotion
test, giving the BMS subscore in vehicle treated mice
(n = 8) and NX210 treated mice (n = 7).

| | | | | NX210 | | |
|---|---|---|---|---|---|---|
| | Vehicle | | | 8 mg/kg | | |
| | BMS subscore | | | BMS subscore | | |
| | Mean | SEM | n | Mean | SEM | n |
| D-1 | 11.00 | 0.00 | 8 | 11.00 | 0.00 | 7 |
| D 2 | 0.25 | 0.25 | 8 | 0.00 | 0.00 | 7 |
| D 7 | 0.13 | 0.13 | 8 | 1.14 | 0.46 | 7 |
| D 14 | 1.63 | 0.73 | 8 | 5.00** | 1.27 | 7 |
| D 21 | 2.13 | 0.83 | 8 | 2.57 | 0.84 | 7 |
| D 28 | 2.75 | 0.94 | 8 | 3.43 | 0.57 | 7 |
| D 35 | 1.63 | 0.63 | 8 | 4.86** | 0.96 | 7 |
| D 42 | 1.25 | 0.70 | 8 | 4.43* | 0.87 | 7 |

*p < 0.05,
**p < 0.01.
Two-way ANOVA followed by Bonferroni's post hoc test.

TABLE 3
Assessing recovery of hindlimb function after a T8 dorsal
hemisection (spinal cord injury) using open field locomotion
test in vehicle treated mice (n = 8) and NX210 treated
mice (n = 7). Percentage of mice with BMS score ≥5.

| | |
|---|---|
| | NX210 |
| Vehicle | 8 mg/kg |

| | BMS ≥ 5 | n | BMS ≥ 5 | n |
|---|---|---|---|---|
| D 7 | 0.00% | 8 | 28.57% | 7 |
| D 14 | 12.50% | 8 | 85.71% | 7 |
| D 21 | 50.00% | 8 | 71.43% | 7 |
| D 28 | 62.50% | 8 | 100.00% | 7 |
| D 35 | 62.50% | 8 | 85.71% | 7 |
| D 42 | 37.50% | 8 | 100.00% | 7 |

TABLE 4
Motor performance after a T8 dorsal hemisection (spinal cord
injury) using rotarod test (retention time on the rotarod)
in vehicle treated mice (n = 8) and NX210 treated mice
(n = 7). Two-way ANOVA followed by Bonferroni's post hoc test

| | | | | NX210 | | |
|---|---|---|---|---|---|---|
| | Vehicle | | | 8 mg/kg | | |
| | Rotarod Time (s) | | | Rotarod Time (s) | | |
| | Mean | SEM | n | Mean | SEM | n |
| D −1 | 66.38 | 6.54 | 8 | 65.64 | 4.39 | 7 |
| D 2 | 6.38 | 1.23 | 8 | 19.29 | 4.73 | 7 |
| D 7 | 24.92 | 5.79 | 8 | 26.95 | 7.23 | 7 |
| D 14 | 19.58 | 3.60 | 8 | 31.19 | 6.31 | 7 |
| D 21 | 23.33 | 3.72 | 8 | 42.33 | 8.56 | 7 |
| D 28 | 21.29 | 4.14 | 8 | 35.05 | 7.61 | 7 |
| D 35 | 15.54 | 3.52 | 8 | 29.95 | 5.60 | 7 |
| D 42 | 17.33 | 5.31 | 8 | 22.52 | 4.84 | 7 |

TABLE 5
Assessing myelin basic protein (MBP) staining intensity after
a T8 dorsal hemisection (spinal cord injury) 1000 μm rostral
and 1000 μm caudal to the injury using immunofluorescence
in vehicle treated mice (n = 3) and NX210 treated mice (n = 5).

| | | | | NX210 | | |
|---|---|---|---|---|---|---|
| | Vehicle | | | 8 mg/kg | | |
| | MBP intensity | | | MBP intensity | | |
| | Mean | SEM | n | Mean | SEM | n |
| 1000-800 | 1190.97 | 258.29 | 3 | 1861.63 | 202.67 | 5 |
| 800-600 | 1198.86 | 262.65 | 3 | 1859.43 | 194.95 | 5 |
| 600-400 | 1221.73 | 243.61 | 3 | 1928.08 | 224.14 | 5 |
| 400-200 | 1100.70 | 192.42 | 3 | 1780.76 | 235.42 | 5 |
| 200-0 | 756.78 | 96.57 | 3 | 1229.59 | 153.51 | 5 |
| 0-200 | 701.71 | 146.17 | 3 | 1292.41 | 168.39 | 5 |
| 200-400 | 778.41 | 179.12 | 3 | 1633.44 | 193.53 | 5 |
| 400-600 | 794.82 | 193.89 | 3 | 1945.87 | 214.97 | 5 |
| 600-800 | 829.76 | 206.61 | 3 | 1987.64 | 189.21 | 5 |
| 800-1000 | 907.40 | 229.69 | 3 | 1892.23 | 145.30 | 5 |

### Model of Spinal Cord Injury—T8 Dorsal Hemisection—Experiment 2

After a T8 dorsal hemisection in mice, NX210 peptide was administered twice a week via intraperitoneal (i.p.) route at different doses (4, 8 and 16 mg/kg), the first injection being performed 4 h post hemisection (4 h post-injury in mice could potentially represent a therapeutic window of one to several days in human). The locomotor function in NX210- or vehicle-treated mice were assessed weekly using the Basso Mouse Scale (BMS) open field test (Tables 6, 7 & 8—FIGS. 4 & 5), the Rotarod test to analyze the performance under forced movement (Tables 8) and activity chamber test to examine spontaneous locomotor activity (Tables 10 & 11). Post-mortem analysis was also performed using immunostainings.

Analysis of locomotor activity using the BMS open field test revealed a significant increased recovery of mice treated with NX210 when compared to vehicle-injected mice (Table 6 & FIG. 6), with a BMS score significantly higher in mice treated with 8 mg/kg or 16 mg/kg of NX210 from day 7 or day 21 respectively, until the end of the study (day 56 post-injury). Mice treated with 4 mg/kg also displayed a higher BMS score than vehicle-injected mice.

Also, BMS subscore was significantly higher in NX210-treated groups at 8 mg/kg or 16 mg/kg from day 21 until the end of the study (day 56 post-injury) (Table 7& FIG. 7). Mice treated with 4 mg/kg also displayed a higher BMS subscore than vehicle-injected mice.

Twenty-five percent (25%) of vehicle-treated mice presented a BMS score ≥5 at the end of the study (at day 56 post-injury) whereas 86% of NX210-treated mice (8 mg/kg) reached a BMS score ≥5 as early as day 21 post-injury and kept until the end of the study, demonstrating strong functional recovery (Table 7). A BMS score ≥5 was reached by 44% and 75% of mice treated with 4 mg/kg or 16 mg/kg of NX210 respectively.

Analysis of locomotor activity using the rotarod test showed that NX210 administrations at 8 mg/kg significantly increased the duration time on the rod, a significant improvement as early as day 8 post-injury and kept until the end of the study (day 57 post-injury) (Table 9). Mice treated with 16 mg/kg of NX210 also presented an increase in duration time on the rotarod in contrast to vehicle-treated mice.

Spontaneous locomotor activity was assessed weekly using activity chamber test. NX210-treated mice (in particular in 8 and 16 mg/kg groups) displayed significant increased total traveled distance and average velocity compared to vehicle-treated mice (Tables 10 & 11), as further demonstrations of functional recovery of NX210-treated animals.

Body weight of NX210-treated mice was increased from day 2 post-injury, reached pre-injury values between day 20 and day 27 and kept increasing until the end of the study, whereas body weight of vehicle-treated-mice only increased from day 27 (slowly) but never reached pre-injury values even at the end of the study (day 58 post-injury), mice also regained weight faster and looked generally healthier (data not shown).

All together, these data confirmed the strong improvement of functional recovery following administrations of NX210, an SCO-Spondin derived peptide, after Spinal Cord Injury.

TABLE 6
Assessing recovery of hindlimb function after a T8 dorsal hemisection
(spinal cord injury) using open field locomotion test, giving the
BMS score in vehicle treated mice (n = 8) and mice treated with
4 mg/kg (n = 9), 8 mg/kg (n = 7) and 16 mg/kg (n =
8). *: p < 0.05, **: p < 0.01. Two-way ANOVA followed by Bonferroni's post hoc test.

| | | | | NX210 | | | NX210 | | | NX210 | | |
|---|---|---|---|---|---|---|---|---|---|---|---|---|
| | Vehicle | | | 4 mg/kg | | | 8 mg/kg | | | 16 mg/kg | | |
| | BMS score | | | BMS score | | | BMS score | | | BMS score | | |
| | Mean | SEM | n | Mean | SEM | n | Mean | SEM | n | Mean | SEM | n |
| D −1 | 9.00 | 0.00 | 8 | 9.00 | 0.00 | 9 | 8.57 | 0.43 | 7 | 9.00 | 0.00 | 8 |
| D 2 | 1.19 | 0.60 | 8 | 1.39 | 0.68 | 9 | 3.50 | 1.12 | 7 | 2.31 | 0.73 | 8 |
| D 7 | 1.94 | 0.71 | 8 | 2.56 | 0.88 | 9 | 5.07** | 0.70 | 7 | 4.13 | 0.82 | 8 |
| D 14 | 2.25 | 0.71 | 8 | 4.11 | 0.72 | 9 | 5.50** | 0.49 | 7 | 4.63 | 0.72 | 8 |
| D 21 | 2.50 | 0.73 | 8 | 4.28 | 0.64 | 9 | 5.57** | 0.58 | 7 | 5.44** | 0.89 | 8 |
| D 28 | 2.81 | 0.81 | 8 | 5.22* | 0.64 | 9 | 5.86** | 0.61 | 7 | 5.56* | 0.89 | 8 |
| D 35 | 3.00 | 0.73 | 8 | 4.61 | 0.55 | 9 | 5.93* | 0.66 | 7 | 5.38 | 0.67 | 8 |
| D 42 | 3.13 | 0.60 | 8 | 4.50 | 0.40 | 9 | 6.14* | 0.46 | 7 | 5.75* | 0.75 | 8 |
| D 49 | 2.88 | 0.71 | 8 | 4.50 | 0.49 | 9 | 6.29** | 0.50 | 7 | 6.00** | 0.56 | 8 |
| D 56 | 3.13 | 0.70 | 8 | 4.67 | 0.47 | 9 | 6.29** | 0.34 | 7 | 5.81* | 0.77 | 8 |

TABLE 7
Assessing recovery of hindlimb function after a T8 dorsal hemisection
(spinal cord injury) using open field locomotion test, giving the BMS
subscore in vehicle treated mice (n = 8) and mice treated with
4 mg/kg (n = 9), 8 mg/kg (n = 7) and 16 mg/kg (n = 8).
*: p < 0.05, **: p < 0.01. Two-way ANOVA followed by Bonferroni's post hoc test.

| | | | | NX210 | | | NX210 | | | NX210 | | |
|---|---|---|---|---|---|---|---|---|---|---|---|---|
| | Vehicle | | | 4 mg/kg | | | 8 mg/kg | | | 16 mg/kg | | |
| | BMS subscore | | | BMS subscore | | | BMS subscore | | | BMS subscore | | |
| | Mean | SEM | n | Mean | SEM | n | Mean | SEM | n | Mean | SEM | n |
| D −1 | 11.00 | 0.00 | 8 | 11.00 | 0.00 | 9 | 10.43 | 0.57 | 7 | 11.00 | 0.00 | 8 |
| D 2 | 0.13 | 0.13 | 8 | 0.33 | 0.17 | 9 | 2.14 | 1.39 | 7 | 0.13 | 0.13 | 8 |
| D 7 | 1.13 | 0.99 | 8 | 1.89 | 1.16 | 9 | 4.29 | 1.46 | 7 | 3.75 | 1.41 | 8 |
| D 14 | 0.88 | 0.88 | 8 | 2.33 | 1.07 | 9 | 4.86 | 1.28 | 7 | 3.75 | 1.35 | 8 |
| D 21 | 1.00 | 0.76 | 8 | 2.44 | 1.02 | 9 | 5.29* | 1.06 | 7 | 5.50* | 1.36 | 8 |
| D 28 | 1.25 | 0.98 | 8 | 4.00 | 1.22 | 9 | 6.14** | 1.26 | 7 | 5.63* | 1.40 | 8 |
| D 35 | 1.00 | 1.00 | 8 | 3.00 | 1.09 | 9 | 5.86** | 1.20 | 7 | 5.25* | 1.26 | 8 |
| D 42 | 1.25 | 0.73 | 8 | 2.56 | 0.87 | 9 | 5.57* | 1.17 | 7 | 5.50* | 1.39 | 8 |
| D 49 | 1.38 | 0.89 | 8 | 2.67 | 0.87 | 9 | 6.86** | 0.99 | 7 | 5.38* | 1.25 | 8 |
| D 56 | 1.38 | 0.98 | 8 | 3.00 | 0.90 | 9 | 6.43** | 1.04 | 7 | 5.63* | 1.34 | 8 |

TABLE 8
Assessing recovery of hindlimb function after T8 dorsal hemisection
(spinal cord injury) using open field locomotion test in vehicle treated
mice (n = 8) and mice treated with 4 mg/kg (n = 9), 8 mg/kg
(n = 7) and 16 mg/kg (n = 8). Percentage of mice with BMS score ≥5.

| | | | NX210 | | NX210 | | NX210 | |
|---|---|---|---|---|---|---|---|---|
| | Vehicle | | 4 mg/kg | | 8 mg/kg | | 16 mg/kg | |
| | BMS ≥ 5 | n | BMS ≥ 5 | n | BMS ≥ 5 | n | BMS ≥ 5 | n |
| D 7 | 12.5% | 8 | 22.2% | 9 | 57.1% | 7 | 37.5% | 8 |
| D 14 | 12.5% | 8 | 55.6% | 9 | 71.4% | 7 | 62.5% | 8 |
| D 21 | 25.0% | 8 | 55.6% | 9 | 85.7% | 7 | 75.0% | 8 |
| D 28 | 25.0% | 8 | 55.6% | 9 | 85.7% | 7 | 75.0% | 8 |
| D 35 | 12.5% | 8 | 55.6% | 9 | 85.7% | 7 | 75.0% | 8 |
| D 42 | 12.5% | 8 | 44.4% | 9 | 100.0% | 7 | 75.0% | 8 |
| D 49 | 12.5% | 8 | 44.4% | 9 | 85.7% | 7 | 75.0% | 8 |
| D 56 | 25.0% | 8 | 44.4% | 9 | 85.7% | 7 | 75.0% | 8 |

TABLE 9
Motor performance after a T8 dorsal hemisection (spinal cord injury) using
rotarod test (retention time on the rod), in vehicle treated mice (n =
8) and mice treated with 4 mg/kg (n = 9), 8 mg/kg (n = 7) and
16 mg/kg (n = 8). *: p < 0.05, **: p < 0.01, ***: p <
0.001. Two-way ANOVA followed by Bonferroni's post hoc test.

| | | | | NX210 | | | NX210 | | | NX210 | | |
|---|---|---|---|---|---|---|---|---|---|---|---|---|
| | Vehicle | | | 4 mg/kg | | | 8 mg/kg | | | 16 mg/kg | | |
| | Rotarod Time (s) | | | Rotarod Time (s) | | | Rotarod Time (s) | | | Rotarod Time (s) | | |
| | Mean | SEM | n | Mean | SEM | n | Mean | SEM | n | Mean | SEM | n |
| D −1 | 82.54 | 7.12 | 8 | 93.89 | 5.45 | 9 | 87.95 | 7.03 | 7 | 92.21 | 4.05 | 8 |
| D 2 | 15.12 | 5.50 | 8 | 19.30 | 4.12 | 9 | 28.00 | 7.56 | 7 | 28.42 | 7.20 | 8 |
| D 8 | 20.88 | 4.76 | 8 | 35.56 | 6.74 | 9 | 53.90* | 9.20 | 7 | 38.08 | 7.73 | 8 |
| D 15 | 20.79 | 6.55 | 8 | 38.30 | 7.84 | 9 | 61.29*** | 8.85 | 7 | 47.08 | 8.00 | 8 |
| D 22 | 18.21 | 4.84 | 8 | 30.78 | 7.38 | 9 | 52.33** | 10.46 | 7 | 34.54 | 6.19 | 8 |
| D 29 | 17.17 | 4.41 | 8 | 32.78 | 7.07 | 9 | 54.29** | 7.33 | 7 | 42.12 | 4.68 | 8 |
| D 36 | 21.25 | 5.50 | 8 | 33.37 | 7.10 | 9 | 54.76** | 9.00 | 7 | 48.25 | 5.50 | 8 |
| D 43 | 16.71 | 4.83 | 8 | 35.85 | 10.38 | 9 | 55.90** | 11.50 | 7 | 40.42 | 8.47 | 8 |
| D 50 | 18.33 | 5.94 | 8 | 42.81 | 10.09 | 9 | 52.38** | 7.41 | 7 | 40.79 | 7.88 | 8 |
| D 57 | 16.21 | 4.24 | 8 | 28.93 | 4.50 | 9 | 49.71** | 8.34 | 7 | 38.29 | 9.26 | 8 |

TABLE 10
Total distance traveled (m) after T8 dorsal hemisection using open-field
test (chamber activity) in vehicle treated mice (n = 8) and mice
treated with 4 mg/kg (n = 9), 8 mg/kg (n = 7) and 16 mg/kg
(n = 8). *p < 0.05. Two-way ANOVA followed by Bonferroni's post hoc test.

| | | | | NX210 | | | NX210 | | | NX210 | | |
|---|---|---|---|---|---|---|---|---|---|---|---|---|
| | Vehicle | | | 4 mg/kg | | | 8 mg/kg | | | 16 mg/kg | | |
| | Total distance | | | Total distance | | | Total distance | | | Total distance | | |
| | traveled (m) | | | traveled (m) | | | traveled (m) | | | traveled (m) | | |
| | Mean | SEM | n | Mean | SEM | n | Mean | SEM | n | Mean | SEM | n |
| D −1 | 37.20 | 3.77 | 8 | 35.14 | 2.03 | 9 | 34.76 | 1.70 | 7 | 37.89 | 2.19 | 8 |
| D 2 | 19.88 | 1.42 | 8 | 24.11 | 2.01 | 9 | 25.92 | 1.86 | 7 | 23.94 | 1.97 | 8 |
| D 8 | 28.34 | 2.36 | 8 | 30.48 | 1.72 | 9 | 34.33 | 2.11 | 7 | 35.19 | 2.18 | 8 |
| D 15 | 28.16 | 2.29 | 8 | 28.73 | 1.79 | 9 | 32.76 | 1.33 | 7 | 32.33 | 2.09 | 8 |
| D 22 | 28.19 | 2.51 | 8 | 29.27 | 2.56 | 9 | 39.26 | 3.17 | 7 | 34.82 | 2.65 | 8 |
| D 29 | 28.47 | 2.31 | 8 | 33.15 | 3.93 | 9 | 40.01* | 4.03 | 7 | 37.55 | 2.52 | 8 |
| D 36 | 29.98 | 2.50 | 8 | 33.67 | 2.65 | 9 | 40.48 | 5.72 | 7 | 40.03 | 2.44 | 8 |
| D 43 | 23.88 | 2.00 | 8 | 33.73 | 3.98 | 9 | 36.19* | 3.68 | 7 | 36.70* | 3.51 | 8 |
| D 50 | 29.49 | 2.27 | 8 | 32.38 | 1.99 | 9 | 41.65* | 5.65 | 7 | 42.02* | 3.37 | 8 |
| D 57 | 25.56 | 2.59 | 8 | 31.84 | 3.09 | 9 | 34.79 | 5.05 | 7 | 38.18* | 4.53 | 8 |

TABLE 11
Average velocity (m/min) after T8 dorsal hemisection using open-
field test (chamber activity) in vehicle treated mice (n =
8) and mice treated with 4 mg/kg (n = 9), 8 mg/kg (n =
7) and 16 mg/kg (n = 8). *p < 0.05, **p < 0.01.
Two-way ANOVA followed by Bonferroni's post hoc test.

| | | | | NX210 | | | NX210 | | | NX210 | | |
|---|---|---|---|---|---|---|---|---|---|---|---|---|
| | Vehicle | | | 4 mg/kg | | | 8 mg/kg | | | 16 mg/kg | | |
| | Average | | | Average | | | Average | | | Average | | |
| | velocity (m/min) | | | velocity (m/min) | | | velocity (m/min) | | | velocity (m/min) | | |
| | Mean | SEM | n | Mean | SEM | n | Mean | SEM | n | Mean | SEM | n |
| D −1 | 4.95 | 0.38 | 8 | 4.60 | 0.18 | 9 | 4.52 | 0.13 | 7 | 4.85 | 0.14 | 8 |
| D 2 | 3.09 | 0.14 | 8 | 3.32 | 0.14 | 9 | 3.46 | 0.14 | 7 | 3.37 | 0.10 | 8 |
| D 8 | 3.75 | 0.14 | 8 | 3.88 | 0.16 | 9 | 4.15 | 0.20 | 7 | 4.18 | 0.18 | 8 |
| D 15 | 3.75 | 0.16 | 8 | 3.74 | 0.16 | 9 | 4.26 | 0.17 | 7 | 3.95 | 0.18 | 8 |
| D 22 | 3.68 | 0.17 | 8 | 3.99 | 0.17 | 9 | 4.67* | 0.25 | 7 | 4.31 | 0.20 | 8 |
| D 29 | 3.69 | 0.18 | 8 | 4.18 | 0.31 | 9 | 4.64 | 0.35 | 7 | 4.52 | 0.19 | 8 |
| D 36 | 3.72 | 0.18 | 8 | 4.02 | 0.23 | 9 | 4.69* | 0.52 | 7 | 4.52 | 0.24 | 8 |
| D 43 | 3.43 | 0.15 | 8 | 4.12 | 0.33 | 9 | 4.32 | 0.32 | 7 | 4.42* | 0.30 | 8 |
| D 50 | 3.65 | 0.19 | 8 | 4.00 | 0.15 | 9 | 4.72* | 0.52 | 7 | 4.82** | 0.34 | 8 |
| D 57 | 3.40 | 0.18 | 8 | 3.92 | 0.21 | 9 | 4.19 | 0.46 | 7 | 4.48* | 0.35 | 8 |

### EXAMPLE 3: NX210 AND NX218 PROTECTION OF RAT PRIMARY CORTICAL NEURONS AGAINST GLUTAMATE-INDUCED EXCITOTOXICITY

Neuronal death by glutamate excitotoxicity is a common pathological feature in SCI. To investigate the neuroprotective potential of NX210 and NX218 (NX210 peptide oxidized or cyclic form) co-treatment experiments with glutamate were performed on rat cortical neurons cultured in vitro and neuronal survival and neurite network were assessed by immunohistochemistry.

### Primary Cultures of Rat Neurons:

Cortical neurons were cultured as described previously (Callizot N, Combes M, Steinschneider R, Poindron P (2013) Operational dissection of β-amyloid cytopathic effects on cultured neurons. J Neurosci Res 91:706-716). Briefly, fetuses were isolated from Wistar rats at gestational day 15, and immediately placed in ice-cold L15 Leibovitz medium containing a 2% penicillin (10,000 U/mL) and streptomycin (10 mg/mL) (PS) solution and 1% bovine serum albumin (Dutscher). Tissues were enzymatically dissociated using 0.05% trypsin and 0.02% ethylene diamine tetraacetic acid (Dutscher) for 20 minutes at 37° C. The effect of trypsin was neutralized by addition of fresh culture medium containing Dulbecco's modified Eagle's medium, 4.5 g/liter of glucose, 0.5 mg/ml DNAse I grade II and 10% fetal calf serum (FCS; Dutscher). Cells were then mechanically dissociated by three forced passages through a 10-mL tip, before being centrifuged at 515 g for 10 minutes at 4° C. The pellet was resuspended in Neurobasal™ medium containing 2% of B27 supplement (Fisher Scientific), 2 mM of L-glutamine (Dutscher), 2% of PS solution, and 10 ng/mL of brain-derived neurotrophic factor (Dutscher). Neurons were finally seeded on 96-well plates at a density of 25,000 cells per well previously coated with poly-L-lysine and cultured at 37° C. in 5% CO2 incubator. The medium was changed every other day. On day 13 of culture, neurons were exposed simultaneously to glutamate (Sigma-Aldrich) at 20 μM and either vehicle (sterile water for cell culture; Dutscher), NX210 or NX218 at 100, 250 or 500 μg/mL for 20 minutes.

### Immunofluorescence of Rat Cortical Neurons:

Forty-eight hours after glutamate exposure, neurons were fixed with a cold solution of ethanol (95%) and acetic acid (5%) for 5 minutes at −20° C., and permeabilized with a solution containing 0.1% of saponin (VWR) in phosphate buffered saline (PBS; Dutscher). Neurons were then incubated with mouse monoclonal anti-microtubule associated protein-2 (MAP-2, 1/400; Sigma-Aldrich) primary antibodies diluted in PBS containing 1% FCS and 0.1 saponin for 2 hours at room temperature.

The number of MAP-2-positive neurons was measured to assess neuronal survival, whereas the cumulative length of MAP-2-positive neurites was measured to assess neurite network. Results are presented in table 12 below and FIG. 6.

TABLE 12
NX210 and NX218 peptides protect rat cortical neurons
against glutamate-induced excitotoxicity in vitro

| | Neuronal survival | | | Neurite length | | |
|---|---|---|---|---|---|---|
| | Mean | SEM | n | Mean | SEM | n |
| control | 100 | 2.08 | 6 | 100 | 4.56 | 6 |
| glutamate (glu) 20 μM | 70.6 | 2.31 | 5 | 63.07 | 0.89 | 6 |
| glu + NX210 100 μg/mL | 72.78 | 3.13 | 5 | 67.28 | 2.66 | 6 |
| glu + NX218 100 μg/mL | 77.5 | 1.91 | 5 | 85.17 | 1.95 | 6 |
| glu + NX210 250 μg/mL | 78.18 | 3.66 | 6 | 77.5 | 1.757 | 6 |
| glu + NX218 250 μg/mL | 85.28 | 3.17 | 6 | 94.88 | 5.365 | 5 |
| glu + NX210 500 μg/mL | 81 | 1.9 | 6 | 74.34 | 3.37 | 5 |
| glu + NX218 500 μg mL | 82.92 | 3.83 | 6 | 85.34 | 3.11 | 5 |

NX218 protected rat cortical neurons from glutamate-induced neuronal death at 250 and 500 μg/mL (29.40% cell death for glu-treated neurons vs 14.72% for NX218/glu-treated neurons at 250 μg/mL, p=0.0101) and fully restored neurite network whatever the dose used (−36.93% total neurite network length for glu-treated neurons vs −5.12% for NX218/glu-treated neurons at 250 μg/mL, p=0.0002). Although NX210 did not display any protective effect on neurite network (p=0.6602, 0.0617 and 0.1487 between glu-treated neurons and NX210/glu-treated neurons at 100, 250 and 500 μg/mL), its highest dose increased neuronal survival (29.40% cell death for glu-treated neurons vs 19.00% for NX210/glu-treated neurons, p=0.0498). Accordingly, neurite network was significantly more preserved with NX218 than NX210 at 100 and 250 μg/mL (p=0.0071, 0.0467 and 0.1419 between glu-treated neurons exposed to NX210 or NX218 at 100, 250 and 500 μg/mL, respectively).

### EXAMPLE 4: EFFECT OF NX210 AND NX218 ON WHITE MATTER REMYELINATION AFTER FOCAL LESION IN THE CORPUS CALLOSUM IN MICE

The therapeutic effects of a post-treatment with two subcommissural organ (SCO)-spondin derived peptides, namely NX210 and its cyclic form NX218 were evaluated on white matter lesion evolution and remyelination after a focal lesion of the corpus callosum (CC) in adult male C57BL/6J mice using magnetic resonance imaging (MRI) and immunohistochemical analyses.

Focal unilateral lesion of the right CC was induced by a stereotaxic injection of lysolecithin (LPC) in adult male C57BL/6J mice. In this model, LPC injection induces a demyelination of axons resulting in a reproducible lesion lasting for more than 21 days in mice (Leonetti et al. Molecular Neurodegeneration, 2017, incorporated herein by reference). The demyelinated volume progressively reduces over time as new oligodendrocyte progenitor cells (OPCs) proliferate, migrate, differentiate into mature oligodendrocytes and remyelinate the lesioned area.

NX210 and NX218 were administered every other day from Day 2 (D2) up to D21 at the dose of 5 mg/kg via intraperitoneal route. Lesion volume for 7-8 mice per group were measured via longitudinal magnetic resonance imaging (MRI) examination with acquisitions at D1, D3, D7, D14 and D21. On D1 before treatment initiation, the mean lesion volume of all groups and subgroups was similar and close to 0.8 mm³. During the first week after LPC injection, the average lesion volume increased in the vehicle-treated group at least until D7, it increased only until D3 in the NX210-treated group, while it remained stable until D3 and then decreased from D3 to D7 in NX218-treated group: at day 7, the average lesion volume was of 0.73 mm³ and 0.75 mm³ in animals treated with NX218 or NX210 respectively versus 0.84 mm³ in vehicle-treated mice. After the first week, the mean lesion volume continuously decreased in all groups until D21 (Table 13 & FIG. 7).

TABLE 13
Lesion volume follow-up after lysolecithin injection
using Magnetic Resonance Imagery (MRI) in vehicle-treated
mice (n = 7) and mice treated with 5 mg/kg of either
NX210 (n = 8) or NX218 (n = 8). Two-way ANOVA
followed by Bonferroni's post hoc test.

| | VEHICLE | | | NX210 (5 mg/kg) | | | NX218 (5 mg/kg) | | |
|---|---|---|---|---|---|---|---|---|---|
| | Lesion volume | | | Lesion volume | | | Lesion volume | | |
| | (mm³ - MRI) | | | (mm³ - MRI) | | | (mm³ - MRI) | | |
| | Mean | SEM | n | Mean | SEM | n | Mean | SEM | n |
| D 1 | 0.770 | 0.047 | 7 | 0.775 | 0.073 | 8 | 0.769 | 0.071 | 8 |
| D 3 | 0.835 | 0.051 | 7 | 0.829 | 0.053 | 8 | 0.766 | 0.036 | 8 |
| D 7 | 0.840 | 0.072 | 7 | 0.746 | 0.069 | 8 | 0.726 | 0.052 | 8 |
| D 14 | 0.698 | 0.062 | 7 | 0.635 | 0.079 | 8 | 0.623 | 0.060 | 8 |
| D 21 | 0.448 | 0.049 | 7 | 0.521 | 0.084 | 8 | 0.401 | 0.038 | 8 |

Myelin Binding Protein (MBP) immunohistochemistry (using the method and antibodies described in Leonetti supra) for 3 vehicle mice at D1, and for 3 animals per group at D3, D7, D14 and D21 were performed to visualize myelin and to measure lesion volume. Oligodendrocyte transcription factor 2 (Olig2) labelling and positive cell counting in the lesion area were also performed for 3 animals per group at D7.

Regarding the lesion area (in percentage of the ipsilateral CC) using MBP immunostaining, the time evolution was similar to the one observed with the MRI lesion evolution with a lesion area increasing until D7 in the vehicle-treated group while already decreasing after D3 in the NX218-treated group. There was a tendency toward smaller lesion area among NX218-treated animals. If analyzed time by time, the lesion area was significatively smaller in NX218-treated group compared to vehicle-treated group on D7 (t-test; p=0.0104) confirming the tendency observed with MRI. (Table 14 & FIG. 8).

TABLE 14
Assessing lesion area after lysolecithin injection using Myelin Binding
Protein (MBP) immunostaining in vehicle-treated mice (n = 3 per
timepoint) and mice treated with 5 mg/kg of either NX210 (n = 3
per timepoint) or NX218 (n = 3 per timepoint). Lesion area expressed
as a percentage of the ipsilateral corpus callosum (CC). *: p < 0.05, t-test.

| BEFORE | | | | | | | | | | | |
|---|---|---|---|---|---|---|---|---|---|---|---|
| TREATMENT | | | VEHICLE | | | NX210 (5 mg/kg) | | | NX218 (5 mg/kg) | | |
| MBP lesion area | | | MBP lesion area | | | MBP lesion area | | | MBP lesion area | | |
| (% of the ipsilateral | | | (% of the ipsilateral | | | (% of the ipsilateral | | | (% of the ipsilateral | | |
| CC) | | | CC) | | | CC) | | | CC) | | |
| Mean | SEM | n | Mean | SEM | n | Mean | SEM | n | Mean | SEM | n |
| 27.6 | 4.6 | 3 | | | | | | | | | |
| | | | 26.0 | 2.1 | 3 | 28.6 | 3.1 | 3 | 24.4 | 2.6 | 2 |
| | | | 33.7 | 1.4 | 3 | 34.3 | 1.1 | 3 | 20.7* | 6.4 | 3 |
| | | | 25.6 | 1.8 | 3 | 32.7 | 2.3 | 3 | 24.4 | 0.3 | 3 |
| | | | 19.3 | 3.2 | 3 | 24.6 | 5.1 | 3 | 17.2 | 3.3 | 3 |

Analysis of Olig2-positive cells was also performed using the method and reagents described in Leonetti supra.

At D7, the density of Olig2-positive cells was significantly higher in the lesion of NX218 animals compared to the vehicle animals (t-test, p=0.028) with respective mean densities per mm² of 8085.9±933.9 and 5007.2±618.4 (Mean±SEM) indicating a higher Olig2 progenitor recruitment in the lesion among NX218-treated animals compared to vehicle-treated animals. These data indicate that NX218 induces OPC recruitment or proliferation or facilitate their migration to the lesion area after demyelination of the CC. NX210 trends to increase this parameter (mean densities per mm² of 7699.9±2026.6) compare the vehicle. There was also no significant difference between vehicle, NX210 and NX218 regarding the Olig2 cell density in the whole ipsilateral- or contralateral CC (Table 15 & FIG. 9).

TABLE 15
Assessing the Olig2-positive cell density in the corpus callosum (CC) after
lysolecithin injection using Olig2 immunostaining in vehicle-treated mice
(n = 3 per timepoint) and mice treated with 5 mg/kg of either NX210
(n = 3 per timepoint) or NX218 (n = 3 per timepoint). *: p < 0.05, t-test.

| | VEHICLE | | | NX210 (5 mg/kg) | | | NX218 (5 mg/kg) | | |
|---|---|---|---|---|---|---|---|---|---|
| | Olig2 cell density | | | Olig2 cell density per | | | Olig2 cell density per | | |
| | per mm² | | | mm² | | | mm² | | |
| | Mean | SEM | n | Mean | SEM | n | Mean | SEM | n |
| CC Ipsilateral | 5401.5 | 822.9 | 3 | 6426.8 | 866.5 | 3 | 5359.0 | 1233.1 | 3 |
| Lesion | 5007.2 | 618.4 | 3 | 7699.9 | 2026.6 | 3 | 8085.9* | 933.9 | 3 |
| CC Contralateral | 5651.4 | 521.9 | 3 | 8050.6 | 1921.0 | 3 | 7964.2 | 923.2 | 3 |

### EXAMPLE 5: PHARMACOKINETICS IN ANIMALS

Preliminary in vitro experiments demonstrated that NX210 is rapidly converted into NX218 by oxidation in rat plasma. Therefore, NX210 PK in animals is followed by the measurement of its cyclic form NX218. First a preliminary PK study in rat was performed in order to validate the method for detecting NX218 in plasma then translated to monkey by performing a more robust PK study with repeated experiments. All PK studies were performed with NaCl 0.9% as vehicle.

Preliminary PK Study in Rat:

In the 4 rats tested, NX218 rapidly decreased in concentration and became impossible to quantify from 3 hours after slow bolus IV injection of 49 mg/kg of NX210 (data not shown). This study demonstrated identification of NX218 in animal plasma was feasible.

PK Study in Monkey:

In the 3 monkeys tested, data were reproducible after repeated testing at different days (D22, D37 and D51), after bolus IV injection of 10 mg/kg of NX210. The concentration of NX218 rapidly decreased up to 30 minutes after the injection (FIG. 10). The half-life was assessed to approximately 12 minutes (Table 16), so in the same range as the one observed for rat and dog (data not shown), with a high consistency for repeated administrations.

TABLE 16
PK data after NX210 IV injection in monkey. AUC: Area Under Curve, CL: total clearance,
Cmax: maximum concentration, t½: terminal elimination half-life,
Tmax: time to reach Cmax, Vss: Volume of distribution at steady state

| | Cₘₐₓ | Tₘₐₓ | AUC₀₋ₜ | AUC_{0-inf} | CL | t_{1/2} | Vₛₛ |
|---|---|---|---|---|---|---|---|
| Study Day | (ng/mL) | (min) | (min*ng/mL) | (min*ng/mL) | (mL/min/kg) | (min) | (mL/kg) |
| 22 (4300), 37 | 19300 | 0 | 57600 | 63900 | 190 | 12.7 | 93.6 |
| (4301 and 4302) | | | | | | | |
| or 51 (4300, | | | | | | | |
| 4301 and 4302) | | | | | | | |

Summary statistics of mean monkey plasma PK parameters for NX218 (±standard deviation when available)

### EXAMPLE 6: NX218 PROTECTION OF HUMAN PRIMARY CORTICAL NEURONS AGAINST GLUTAMATE-INDUCED EXCITOTOXICITY

To confirm NX218 (NX210 peptide oxidized or cyclic form) neuroprotective potential on human cortical neurons, co-treatment experiment with glutamate were performed on human neuronal cell cultures and neuron survival and neurite network were assessed using several assays. Results are presented in Table 17-19 below.

### Material and Methods:

Primary culture of human cortical neurons: Fetal human cortical neurons (Sciencell Research Laboratories) were seeded on 96-well plates at a density of 30,000 cells per well previously coated with 1 mg/mL poly-L-lysine (Sigma-Aldrich) and cultured in Neurobasal™ medium containing 2% of B27 supplement (ThermoFisher) at 37° C. in 5% CO2 incubator. On the next day, the culture medium was changed to remove residual dimethyl sulfoxide (Sigma-Aldrich) and unattached cells. Thereafter, the culture medium was changed every other day, up to 7 days in vitro (div). At 8 div, neurons were exposed simultaneously to 100 μM glutamate (Sigma-Aldrich) and either vehicle or NX218 at 100, 250 or 500 μg/mL in a culture medium deprived of B27 supplement for 15 minutes. Different culture plates were used to perform on one hand lactate dehydrogenase (LDH) and neuron-specific class III beta-tubulin (Tuj1) immunostaining and on the other hand WST-8 assays and caspases 3/7 staining, as described below.

WST-8 assay: Twenty-four hours after glutamate exposure, the viability of human neurons was assessed by measuring the reduction of WST-8 to formazan (Sigma-Aldrich). For that purpose, neurons were incubated with 10 μL of CCK-8 reagent (WST-8) for 1 hour at 37° C. before quantifying absorbance at 450 nm using the Synergy II microplate reader. Data are expressed as a percentage of the absorbance in the cell layers of the vehicle control.

LDH assay: Twenty-four hours after glutamate exposure, the plasma membrane integrity of human neurons was assessed by measuring LDH release in culture supernatants using the “Cytotoxicity detection kit (LDH)” (Roche). For that purpose, neurons were incubated with sodium pyruvate in the presence of nicotinamide adenine dinucleotide hydrogen (NADH). Pyruvic acid was catalyzed into lactic acid by free LDH along with a simultaneous oxidation of NADH to NAD+. The rate of oxidation of NADH to NAD+ was measured at 490 nm using the Synergy II microplate reader. Data are expressed as a percentage of LDH content in the culture media of the vehicle control.

Immunofluorescence of human neurons: Twenty-four hours after glutamate exposure, neurons were fixed with 4% of paraformaldehyde (Sigma-Aldrich) in PBS. Then, non-specific sites were blocked with 3% BSA in PBS (Santa Cruz). Cells were incubated with mouse anti-Tuj1 antibody (1/1000; Abcam) diluted in blocking buffer for 1 hour at RT. After several washes, cells were then incubated with an anti-mouse Alexa fluor-488 conjugated secondary antibody (1/100; Abcam) diluted in 0.5% BSA in PBS for 1 hour at RT. Four pictures per well were acquired at 10× magnification for each condition using the CellInsight CX7 fluorescent microscope (ThermoFisher). Image analyses were performed using Cellomics analyzer system (ThermoFisher) to measure several neurite growth parameters including the average length of neurites, and root and extremity numbers. Data are expressed as a percentage of the vehicle control.

Caspase 3/7 assay: Twenty-four hours after glutamate exposure, activation of caspases 3 and 7 was determined by the addition of a fluorogenic substrate of caspases 3/7 to the culture medium (Cell event Caspase 3/7 green detection kit; ThermoFisher). After several washes, four images per well were acquired at 10× magnification with the CellInsight CX7 fluorescent microscope and analyzed with Cellomics analyzer system. Data are expressed as a percentage of caspases 3/7-positive neurons over the total number of nuclei.

TABLE 17-19
Assessing neuronal survival, death and apoptosis and neurite network in human
primary cortical neuronal cultures two days after co-exposure with glutamate
(glu,100 μM), and with either vehicle or NX218 (100, 250, 500 μg/mL)
for 15 minutes at 8 days in vitro. Primary cortical neurons isolated from human
fetuses were co-treated with glutamate (glu, 100 μM), and either vehicle
(control) or NX218 (100, 250, 500 μg/mL) for 15 minutes at 8 day in vitro
(div). One day later, cultures were either subjected to biochemical assays
(WST-8 for the cell layer, lactate dehydrogenase (LDH) for the culture medium),
or to staining with the neuronal marker neuron-specific class III beta-tubulin
(Tuj1) and with caspases 3 and 7 (apoptotic markers). A. Neuron viability was
assessed by WST-8 biochemical assay in the cell layer. B. Neuronal death was
assessed by measuring LDH content in the culture medium. C. The number of apoptotic
cells was assessed by measuring the activation of caspases 3 and 7. Results
are expressed as a percentage of apoptotic neurons over the total number of
nuclei. D-F. Neurite growth was assessed by measuring the average length of
neurites (D), and the number of roots (E) and extremities (F) of Tuj1-positive
neurons. A-F. Data are presented as median and interquartile range. One way-
ANOVA followed by Tukey's multiple comparisons test: ###p <
0.001 control vs glu; ***p < 0.001, **p < 0.01, *p < 0.05 glu
vs NX218/glu, n = 6 (A-E) and n = 5-6 (F).

| | Neuronal survival (A) | | | Neuronal death (B) | | |
|---|---|---|---|---|---|---|
| | Mean | SEM | n | Mean | SEM | n |
| control | 100 | 1.745 | 6 | 100 | 2.55 | 6 |
| glutamate (glu) 100 μM | 77.41^{###} | 1.735 | 6 | 202.5^{###} | 8.416 | 6 |
| glu + NX218 100 μg/mL | 76.71 | 1.431 | 6 | 169.1** | 2.094 | 6 |
| glu + NX218 250 μg/mL | 85.88 | 2.01 | 6 | 149.8*** | 5.85 | 6 |
| glu + NX218 500 μg/mL | 96.94*** | 4.234 | 6 | 160.8*** | 7.599 | 6 |

| | Neuronal apoptosis(C) | | | Neurite length (D) | | |
|---|---|---|---|---|---|---|
| | Mean | SEM | n | Mean | SEM | n |
| control | 14.27 | 0.9454 | 6 | 100 | 6.638 | 6 |
| glutamate (glu) 100 μM | 27.83^{###} | 1.458 | 6 | 36.84^{###} | 7.698 | 6 |
| glu + NX218 100 μg/mL | 18.77*** | 1.221 | 6 | 63.27* | 5.873 | 6 |
| glu + NX218 250 μg/mL | 12.84*** | 1.323 | 6 | 73.27** | 1.597 | 6 |
| glu + NX218 500 μg/mL | 11.03*** | 1.054 | 6 | 51.82 | 5.746 | 6 |

| | Neurite roots (E) | | | Neurite extremities (F) | | |
|---|---|---|---|---|---|---|
| | Mean | SEM | n | Mean | SEM | n |
| control | 100 | 6.082 | 6 | 100 | 7.017 | 6 |
| glutamate (glu) 100 μM | 44.41^{###} | 5.151 | 6 | 35.15^{###} | 5.182 | 6 |
| glu + NX218 100 μg/mL | 69.27* | 5.216 | 6 | 59.25 | 8.638 | 6 |
| glu + NX218 250 μg/mL | 80.18*** | 1.547 | 6 | 67.51* | 0.584 | 5 |
| glu + NX218 500 μg/mL | 61.69 | 6.099 | 6 | 47.79 | 6.611 | 6 |

The neuroprotective effect of NX218 was confirmed in human cortical neurons exposed to glutamate. Indeed, by assessing WST-8 colorimetric assay, it was showed that NX218 fully preserved neuronal viability at 500 μg/mL (−22.59% neuronal viability for glu-treated neurons vs −3.06% for NX218/glu-treated neurons, p<0.0001; p=0.9009 between control and NX218/glu-treated neurons). Moreover, there was significantly less LDH released by neurons co-treated with glutamate and NX218, whatever the dose used, than with glutamate alone (−52.70% LDH in NX218/glu-treated neurons compared to glu-treated neurons at 250 μg/mL, p<0.0001). The beneficial action of NX218 on necrosis was accompanied by a normal basal level of apoptotic cells (14.27% apoptotic cells for control neurons vs 27.83% for glu-treated neurons and 12.84% for NX218/glu-treated neurons at 250 μg/mL, p<0.0001 between control and glu-treated neurons, p<0.0001 between glu-treated and NX218/glu-treated neurons, and p=0.9170 between control and NX218/glu-treated neurons), unravelling a strong neuroprotective effect of NX218 on both necrosis and apoptosis in human cortical neurons.

1.-21. (canceled)

22. A method of treating spinal cord injury in a subject in need thereof, the method comprising:
administering to the subject a therapeutic amount of a peptide of amino acid sequence
(SEQ ID NO: 1)
X1-W-S-A1-W-S-A2-C-S-A3-A4-C-G-X2
in which:
A1, A2, A3 and A4 consists of amino acid sequences consisting of 1 to 5 amino acids,
X1 and X2 consists of amino acid sequences consisting of 1 to 6 amino acids; or X1 and X2 are absent;
it being possible for the N-terminal amino acid to be acetylated, for the C-terminal amino acid to be amidated, or the N-terminal amino acid to be acetylated and the C-terminal amino acid to be amidated,
wherein said peptide of sequence SEQ ID NO: 1 is a linear peptide or an oxidized peptide with the cysteines appearing on the peptide formula of SEQ ID NO: 1 forming a disulfide bridge, or a mixture of both linear and oxidized peptide,
wherein administering the peptide to said subject is through a systemic route.

23. The method of claim 22, wherein the peptide is of amino acid sequence W-S-A1-W-S-A2-C-S-A3-A4-C-G (SEQ ID NO: 2) in which:
A1, A2, A3 and A4 consists of amino acid sequences consisting of 1 to 5 amino acids, wherein said peptide of sequence SEQ ID NO: 2 is a linear peptide or an oxidized peptide with the cysteines appearing on the peptide formula of SEQ ID NO: 2 forming a disulfide bridge, or a mixture of both linear and oxidized peptide.

24. The method of claim 22, wherein
A1 is selected from the group consisting of G, V, S, P and A,
A2 is selected from the group consisting of G, V, S, P and A,
A3 is selected from the group consisting of R, A and V, and/or
A4 is selected from the group consisting of S, T, P and A.

25. The method of claim 23, wherein
A1 is selected from the group consisting of G, V, S, P and A,
A2 is selected from the group consisting of G, V, S, P and A,
A3 is selected from the group consisting of R, A and V, and/or
A4 is selected from the group consisting of S, T, P and A.

26. The method of claim 23, wherein
A1 is G or S,
A2 is G or S,
A3 is R or V, and/or
A4 is S or T.

27. The method of claim 22, wherein A1 and A2 are independently selected from the group consisting of G and S, and/or A3-A4 is selected from the group consisting of R-S, V-S, V-T and R-T.

28. The method of claim 23, wherein A1 and A2 are independently selected from the group consisting of G and S, and/or A3-A4 is selected from the group consisting of R-S, V-S, V-T and R-T.

29. The method of claim 22, wherein the peptide is of a sequence selected from the group consisting of sequences SEQ ID NOS: 3-63.

30. The method of claim 22, wherein the peptide is of sequence SEQ ID NO: 3.

31. The method of claim 22, wherein the peptide is administered to the patient via a route selected from the group consisting of subcutaneous, intravenous, intraperitoneal, intranasal, subcutaneous, intramuscular, sublingual, and oral routes.

32. The method of claim 22, wherein the injury is selected from the group consisting of a traumatic injury and an injury resulting from the growth of surrounding cells.

33. The method of claim 22, which induces inhibition or reducing of neural cell death and/or axonal degeneration, and/or necrosis, and/or secondary injuries.

34. The method of claim 22, which induces myelination.

35. The method of claim 22, which induces functional recovery.

36. The method of claim 22, which induces an increase of the level of myelin binding protein (MBP) at a lesion site.

37. The method of claim 22, which induces an increase of Olig2-positive progenitor recruitment and/or of Olig2-positive cell generation.

38. A method of treating an optic nerve injury in a subject in need thereof, comprising administering to the subject a therapeutic amount of a peptide of amino acid sequence
(SEQ ID NO: 1)
X1-W-S-A1-W-S-A2-C-S-A3-A4-C-G-X2
in which:
A1, A2, A3 and A4 consists of amino acid sequences consisting of 1 to 5 amino acids,
X1 and X2 consists of amino acid sequences consisting of 1 to 6 amino acids; or X1 and X2 are absent;
it being possible for the N-terminal amino acid to be acetylated, for the C-terminal amino acid to be amidated, or the N-terminal amino acid to be acetylated and the C-terminal amino acid to be amidated,
wherein said peptide of sequence SEQ ID NO: 1 is a linear peptide or an oxidized peptide with the cysteines appearing on the peptide formula of SEQ ID NO: 1 forming a disulfide bridge, or a mixture of both linear and oxidized peptide,
wherein administering the peptide to said subject is through a systemic route.

39. A method of treatment of a myelopathy in a patient in need thereof, comprising administering to the patient a therapeutic amount of a peptide of amino acid sequence
(SEQ ID NO: 1)
X1-W-S-A1-W-S-A2-C-S-A3-A4-C-G-X2
in which:
A1, A2, A3 and A4 consists of amino acid sequences consisting of 1 to 5 amino acids,
X1 and X2 consists of amino acid sequences consisting of 1 to 6 amino acids; or X1 and X2 are absent;
it being possible for the N-terminal amino acid to be acetylated, for the C-terminal amino acid to be amidated, or the N-terminal amino acid to be acetylated and the C-terminal amino acid to be amidated,
wherein said peptide of sequence SEQ ID NO: 1 is a linear peptide or an oxidized peptide with the cysteines appearing on the peptide formula of SEQ ID NO: 1 forming a disulfide bridge, or a mixture of both linear and oxidized peptide,
wherein administering the peptide to said subject is through a systemic route.


SEQUENCE LISTING (63 sequences)

SEQ ID NO: 1 - protein, 40 aa, artificial sequence
  Peptide compound
  MISC_FEATURE (1)..(6): Xaa  is absent or 1 amino acid
  MISC_FEATURE (9)..(9): Xaa  is 1 amino acid
  MISC_FEATURE (10)..(13): Xaa  is  absent or 1 amino acid
  MISC_FEATURE (16)..(16): Xaa is 1 amino acid
  MISC_FEATURE (17)..(20): Xaa is absent or 1 amino acid
  MISC_FEATURE (23)..(24): Xaa is 1 amino acid
  MISC_FEATURE (25)..(32): Xaa is absent or 1 amino acid
  MISC_FEATURE (35)..(40): Xaa is  absent or  1 amino acid
XXXXXXWSXXXXXWSXXXXXCSXXXXXXXXXXCGXXXXXX

SEQ ID NO: 2 - protein, 28 aa, Artificial sequence
  Peptide compound
  MISC_FEATURE (3)..(3): Xaa is 1 amino acid
  MISC_FEATURE (4)..(7): Xaa is absent or 1 amino acid
  MISC_FEATURE (10)..(10): Xaa is 1 amino acid
  MISC_FEATURE (11)..(14): Xaa is absent or 1 amino acid
  MISC_FEATURE (17)..(18): Xaa is 1 amino acid
  MISC_FEATURE (19)..(26): Xaa is absent or 1 amino acid
WSXXXXXWSXXXXXCSXXXXXXXXXXCG

SEQ ID NO: 3 - protein, 12 aa, Artificial sequence
  Peptide compound
WSGWSSCSRSCG

SEQ ID NO: 4 - protein, 12 aa, Artificial sequence
  Peptide compound
WSSWSGCSRSCG

SEQ ID NO: 5 - protein, 12 aa, artificial sequence
  peptide compound
WSSWGSCSRSCG

SEQ ID NO: 6 - protein, 12 aa, artificial sequence
  peptide compound
WSSWGGCSRSCG

SEQ ID NO: 7 - protein, 12 aa, artificial sequence
  peptide compound
WSSWSSCSRSCG

SEQ ID NO: 8 - protein, 12 aa, artificial sequence
  peptide compound
WSGWSGCSRSCG

SEQ ID NO: 9 - protein, 12 aa, artificial sequence
  peptide compound
WSGWGSCSRSCG

SEQ ID NO: 10 - protein, 12 aa, artificial sequence
  peptide compound
WSGWGGCSRSCG

SEQ ID NO: 11 - protein, 12 aa, artificial sequence
  peptide compound
WSSWSSCSVSCG

SEQ ID NO: 12 - protein, 12 aa, artificial sequence
  peptide compound
WSSWSGCSVSCG

SEQ ID NO: 13 - protein, 12 aa, artificial sequence
  peptide compound
WSSWGSCSVSCG

SEQ ID NO: 14 - protein, 12 aa, artificial sequence
  peptide compound
WSSWGGCSVSCG

SEQ ID NO: 15 - protein, 12 aa, artificial sequence
  peptide compound
WSGWSSCSVSCG

SEQ ID NO: 16 - protein, 12 aa, artificial sequence
  peptide compound
WSGWSGCSVSCG

SEQ ID NO: 17 - protein, 12 aa, artificial sequence
  peptide compound
WSGWGSCSVSCG

SEQ ID NO: 18 - protein, 12 aa, artificial sequence
  peptide compound
WSGWGGCSVSCG

SEQ ID NO: 19 - protein, 12 aa, artificial sequence
  peptide compound
WSSWSSCSVTCG

SEQ ID NO: 20 - protein, 12 aa, artificial sequence
  peptide compound
WSSWSGCSVTCG

SEQ ID NO: 21 - protein, 12 aa, artificial sequence
  peptide compound
WSSWGSCSVTCG

SEQ ID NO: 22 - protein, 12 aa, artificial sequence
  peptide compound
WSSWGGCSVTCG

SEQ ID NO: 23 - protein, 12 aa, artificial sequence
  peptide compound
WSGWSSCSVTCG

SEQ ID NO: 24 - protein, 12 aa, Artificial Sequence
  peptide compound
WSGWSGCSVTCG

SEQ ID NO: 25 - protein, 12 aa, Artificial Sequence
  peptide compound
WSGWGSCSVTCG

SEQ ID NO: 26 - protein, 12 aa, Artificial Sequence
  peptide compound
WSGWGGCSVTCG

SEQ ID NO: 27 - protein, 12 aa, Artificial Sequence
  peptide compound
WSSWSSCSRTCG

SEQ ID NO: 28 - protein, 12 aa, artificial sequence
  peptide compound
WSSWSGCSRTCG

SEQ ID NO: 29 - protein, 12 aa, Artificial Sequence
  peptide compound
WSSWGSCSRTCG

SEQ ID NO: 30 - protein, 12 aa, Artificial Sequence
  peptide compound
WSSWGGCSRTCG

SEQ ID NO: 31 - protein, 12 aa, Artificial Sequence
  peptide compound
WSGWSSCSRTCG

SEQ ID NO: 32 - protein, 12 aa, Artificial Sequence
  peptide compound
WSGWSGCSRTCG

SEQ ID NO: 33 - protein, 12 aa, Artificial Sequence
  peptide compound
WSGWGSCSRTCG

SEQ ID NO: 34 - protein, 12 aa, Artificial Sequence
  peptide compound
WSGWGGCSRTCG

SEQ ID NO: 35 - protein, 13 aa, Artificial Sequence
  peptide compound
PWSGWSSCSRSCG

SEQ ID NO: 36 - protein, 14 aa, Artificial Sequence
  peptide compound
APWSGWSSCSRSCG

SEQ ID NO: 37 - protein, 15 aa, Artificial Sequence
  peptide compound
LAPWSGWSSCSRSCG

SEQ ID NO: 38 - protein, 16 aa, Artificial Sequence
  peptide compound
VLAPWSGWSSCSRSCG

SEQ ID NO: 39 - protein, 13 aa, Artificial Sequence
  peptide compound
WSGWSSCSRSCGL

SEQ ID NO: 40 - protein, 14 aa, Artificial Sequence
  peptide compound
WSGWSSCSRSCGLG

SEQ ID NO: 41 - protein, 15 aa, Artificial Sequence
  peptide compound
WSGWSSCSRSCGLGL

SEQ ID NO: 42 - protein, 16 aa, Artificial Sequence
  peptide compound
WSGWSSCSRSCGLGLI

SEQ ID NO: 43 - protein, 17 aa, Artificial Sequence
  peptide compound
WSGWSSCSRSCGLGLIF

SEQ ID NO: 44 - protein, 14 aa, Artificial Sequence
  peptide compound
PWSGWSSCSRSCGL

SEQ ID NO: 45 - protein, 15 aa, Artificial Sequence
  peptide compound
PWSGWSSCSRSCGLG

SEQ ID NO: 46 - protein, 16 aa, Artificial Sequence
  peptide compound
PWSGWSSCSRSCGLGL

SEQ ID NO: 47 - protein, 17 aa, Artificial Sequence
  peptide compound
PWSGWSSCSRSCGLGLI

SEQ ID NO: 48 - protein, 18 aa, Artificial Sequence
  peptide compound
PWSGWSSCSRSCGLGLIF

SEQ ID NO: 49 - protein, 15 aa, Artificial Sequence
  peptide compound
APWSGWSSCSRSCGL

SEQ ID NO: 50 - protein, 16 aa, Artificial Sequence
  peptide compound
APWSGWSSCSRSCGLG

SEQ ID NO: 51 - protein, 17 aa, Artificial Sequence
  peptide compound
APWSGWSSCSRSCGLGL

SEQ ID NO: 52 - protein, 18 aa, Artificial Sequence
  peptide compound
APWSGWSSCSRSCGLGLI

SEQ ID NO: 53 - protein, 19 aa, Artificial Sequence
  peptide compound
APWSGWSSCSRSCGLGLIF

SEQ ID NO: 54 - protein, 16 aa, Artificial Sequence
  peptide compound
LAPWSGWSSCSRSCGL

SEQ ID NO: 55 - protein, 17 aa, Artificial Sequence
  peptide compound
LAPWSGWSSCSRSCGLG

SEQ ID NO: 56 - protein, 18 aa, Artificial Sequence
  peptide compound
LAPWSGWSSCSRSCGLGL

SEQ ID NO: 57 - protein, 19 aa, Artificial Sequence
  peptide compound
LAPWSGWSSCSRSCGLGLI

SEQ ID NO: 58 - protein, 20 aa, Artificial Sequence
  peptide compound
LAPWSGWSSCSRSCGLGLIF

SEQ ID NO: 59 - protein, 17 aa, Artificial Sequence
  peptide compound
VLAPWSGWSSCSRSCGL

SEQ ID NO: 60 - protein, 18 aa, Artificial Sequence
  peptide compound
VLAPWSGWSSCSRSCGLG

SEQ ID NO: 61 - protein, 19 aa, Artificial Sequence
  peptide compound
VLAPWSGWSSCSRSCGLGL

SEQ ID NO: 62 - protein, 20 aa, Artificial Sequence
  peptide compound
VLAPWSGWSSCSRSCGLGLI

SEQ ID NO: 63 - protein, 21 aa, Artificial Sequence
  peptide compound
VLAPWSGWSSCSRSCGLGLIF